(19) United States
(12) Patent Application Publication
(10) Pub. No.: US 2015/0173619 A1
(43) Pub. Date: Jun. 25, 2015

(54) ORGAN MAPPING SYSTEM USING AN OPTICAL COHERENCE TOMOGRAPHY PROBE

(71) Applicant: COLLAGE MEDICAL IMAGING LTD., Beer-Sheva (IL)

(72) Inventors: Roni Zvuloni, Haifa (IL); Gavriel J. Iddan, Haifa (IL)

(73) Assignee: COLLAGE MEDICAL IMAGING LTD., Beer-Sheva (IL)

(21) Appl. No.: 14/394,545

(22) PCT Filed: Apr. 17, 2013
(86) PCT No.: PCT/IL2013/050336

§ 371 Date: Dec. 16, 2014

Related U.S. Application Data
(60) Provisional application No. 61/625,151, filed on Apr. 17, 2012.
(60) Provisional application No. 61/625,221, filed on Apr. 17, 2012.

(51) Int. Cl. A61B 5/00; A61B 5/06

(52) U.S. Cl. CPC A61B 5/0066; A61B 5/7282; A61B 5/065; A61B 5/742

(57) ABSTRACT

Systems and methods for scanning an organ or other extended volumes of body tissue using one or more Optical Coherence Tomography (OCT) probes are presented. Some embodiments provide equipment for managing a plurality of OCT penetrations into a tissue or organ, and provide some or all of the following: detection and/or control of OCT probe positions and orientations (and optionally, that of other imaging modalities) detecting changes in body tissue positions, registering and mapping OCT scan results and optionally input from other imaging modalities, integrating OCT scan information and/or information from other modalities and/or recorded historical information, optionally some or all of the above with reference to a common coordinate system. Some embodiments comprise a display for displaying some or all of this information. In some embodiments, inferences based on observed portions of the organ relative to non-observed portions of an organ are displayed.

### RELATED APPLICATION/S

This application claims the benefit of priority under 35 USC §119(e) of U.S. Provisional Patent Application No. 61/625,221 filed Apr. 17, 2012, and of U.S. Provisional Patent Application No. 61/625,151 filed Apr. 17, 2012. The contents of these applications are incorporated herein by reference in their entirety.

### FIELD AND BACKGROUND OF THE INVENTION

The present invention, in some embodiments thereof, relates to a tissue mapping and 3D modeling systems and methods, and, more particularly, but not exclusively, to methods and systems for mapping and modeling an organ using optical coherence tomography (“OCT”).

Optical coherence tomography is an emerging non-invasive optical imaging technique that can be used to perform high-resolution cross-sectional in vivo and in situ imaging of microstructure in materials and in biological tissues.

OCT was first demonstrated by Huang et al. in 1991. U.S. Pat. No. 6,564,087 to Pitris et al. discloses fiber optic needle probes for OCT imaging, as does and U.S. Pat. No. 7,952,718 to Xingde Li et al.

The first clinical applications of OCT were in ophthalmology. Since then, OCT imaging has found uses in a wide range of clinical specialties which involve imaging pathology in tissues that tend to scatter light. Deliverable to the neighborhood of scanned tissues by catheter, by endoscope, by laparoscope, and by needle, OCT promises to have a powerful impact on many medical applications ranging from the screening and diagnosis of neoplasia to enabling new minimally invasive surgical procedures. An article “Real-time three-dimensional optical coherence tomography image-guided core-needle biopsy system”, by Wei-Cheng Kuo, Jongsik Kim, Nathan D. Shemonski, Eric J. Chaney, Darold R. Spillman, Jr., and Stephen A. Boppart, BIOMEDICAL OPTICS EXPRESS, April-June 2012, vol. 3, No. 6, pages 1149-1161, discusses some uses of OCT imaging techniques.

As used in biological/clinical contexts, currently popular versions of OCT probes project towards tissues electromagnetic waves, typically in visible, IR, or Near IR wavelengths. The probe system then typically measures magnitude and “echo time” (the time interval between sending an electromagnetic pulse and detecting an echo) of the electromagnetic waves backscattered from those tissues.

In contrast with sound waves used to generate imaging data in ultrasound probe systems, echo time delays associated with light are extremely fast, indeed too fast to permit direct electronic detection using methods currently known. Consequently OCT probes use methods such as interferometery in analyzing received data. OCT probe systems, projecting light into tissue and using interferometric methods to isolate light reflections and to calculate object distances as indicated by measured echo delays, may achieve image resolutions of 1-15 micrometers, and sub micrometer resolutions have been reported. Such resolutions may be one or two orders of magnitude finer than resolutions achieved by conventional imaging modalities used in the clinical context, such as ultrasound, MRI, and CT. Such high resolutions, available in in vivo contexts, may enable a broad range of research and clinical applications.

Echo time delays associated with light are extremely fast. The measurement of distances with a ˜10 micrometer resolution, which is typical in OCT imaging, requires a time resolution of ˜30 femtoseconds (30×10⁻¹⁵ seconds). Direct electronic detection is not possible on this time scale, but interferometery can detect timing differences on this scale. The most common detection method uses a Michelson interferometer with a scanning reference delay arm. In what is called a “time domain” method of interferometery, a light source, typically a broadband super luminescent diode or a narrow line width laser, provides light directed into the tissues and also along a reference arm. Light reflected/scattered back from the tissues is combined with light reflecting back from the end of the reference arm, and an interference pattern and/or a resultant combined amplitude is detected, from which it is possible to calculate the distance of a reflecting/scattering object as compared to the length of the reference arm. In an alternate “frequency domain” method of use of the OCT, a laser light source is rapidly tuned across a broadband of wavelengths, and Fourier analysis is used to deduce imaged structures at a variety of distances.

In medicine, OCT enables real-time, in situ visualization of tissue microstructure without the need to remove and process specimens. OCT processes may in some contexts enable medical personnel to visualize tissue morphology in situ and in real time, and therefore have been used both for diagnostic imaging and for real-time guidance of surgical intervention.

OCT systems, using implementations of fiber optic technologies together with interferometric techniques, are currently configured for use in catheters and endoscopes which can reach the body organs in a minimally invasive manner, and OCT probes so delivered to near an area of interest in a body can in some cases scan tissues without penetrating them. Alternatively, an OCT probe system such as that taught by Pitris op. cit. may in some cases be used to penetrate tissue and to scan a small tissue volume from within the tissue.

### SUMMARY OF THE INVENTION

The fact that the range of OCT scanning today is only 2-3 mm in highly light-scattering tissue has greatly limited the uses to which OCT scanning has currently been put.

According to methods of prior art, OCT techniques have not previously been used to scan a large volume or an entire organ for diagnostic purposes. Some embodiments of the present invention comprise means and methods for relatively large-scale diagnostic scanning of organs or parts of organs, and for mapping the scanned volume in a three-dimensional reconstructed model, optionally presented on a display, optionally in real time, which enables comparisons with past and future diagnostic information and which may serve as a guide to a therapeutic procedure.

According to an aspect of some embodiments of the present invention there is provided a system for creating a three dimensional map of at least a portion of an organ, comprising:
a) at least one Optical Coherence Tomography (OCT) probe operable to report imaging data while inserted in the organ; and
b) a processor programmed to receive the imaging data during a plurality of tissue insertions of the at least one probe and to record the data with reference to a three-dimensional coordinate system.

According to some embodiments of the invention, the data extends over a three-dimensional volume greater than a volume imageable by a single probe during a single insertion.

According to some embodiments of the invention, the system further comprises a probe location module operable to report location of the at least one OCT probe while the probe is reporting imaging data.

According to some embodiments of the invention, the probe comprises a sensor operable to report position of the probe.

According to some embodiments of the invention, the system further comprises a probe positioning module operable to position the probe at a selected position according to a received command specifying the selected position.

According to some embodiments of the invention, the system further comprises a positioning module operable to guide a plurality of probe insertions to probe positions at predetermined angles and distances one from another.

According to some embodiments of the invention, the positioning module is operable to position the probe for a plurality of sequential insertions into the organ.

According to some embodiments of the invention, the positioning module is operable to insert a plurality of OCT probes into the organ at a same time.

According to some embodiments of the invention, the system further comprises a position reporting module operable to inform a user of a difference between position of a probe positioned by the user and a pre-defined desired position for the probe.

According to some embodiments of the invention, the system further comprises a template which comprises a plurality of guiding channels for guiding the probe during insertion of the probe into the organ.

According to some embodiments of the invention, the system further comprises a second imaging modality in addition to the OCT probe.

According to some embodiments of the invention, the second imaging modality reports location of the at least a portion of the organ to at least one of:
a) a processor; and
b) a display visible by a user.

According to some embodiments of the invention, the system further comprises a position reporting module able to report position of the second imaging modality during imaging operation of the second imaging modality.

According to some embodiments of the invention, the position reporting module comprises a position sensor attached to or in the imaging modality.

According to some embodiments of the invention, the imaging modality is an ultrasound probe which comprises a guide useable to guide insertion of the OCT probe into the organ.

According to some embodiments of the invention, the processer is programmed to analyze image data reported by the probe and to detect, based on the data, an imaged border of the organ.

According to some embodiments of the invention, the system further comprises a servomechanism operable to move the probe, and the processor is further programmed to calculate a command for the servomechanism after the processor detects imaging of the border of the organ.

According to some embodiments of the invention, the processor is operable to control a probe insertion by controlling the servomechanism, and is further operable to command cessation of insertion after analysis of image data from the probe detects a border of the organ.

According to some embodiments of the invention, the processor is operable to control a probe insertion by controlling the servomechanism, and is further programmed change movement of the probe after analysis of image data from the probe detects a suspected lesion in scanned tissue.

According to some embodiments of the invention, the system further comprises an OCT probe also operable to remove a biopsy sample from a body.

According to some embodiments of the invention, the system further comprises a display for displaying an image based on at least a part of a three dimensional mapping created by the system.

According to some embodiments of the invention, the system further comprises a stereoscopic display.

According to some embodiments of the invention, the system further comprises a display calculation module operable to calculate a view based on information from the three dimensional mapping, which information was at least partially calculated based on some of the imaging data.

According to some embodiments of the invention, the system further comprises a display calculation module operable to calculate a view based on information from the three dimensional model, based on information from OCT scanning and information from at least one of
a) a historical data source; and
b) an additional imaging modality, other than OCT scanning.

According to some embodiments of the invention, the calculated view is based on information received by the processor during a plurality of probe tissue insertions.

According to some embodiments of the invention, the calculated view is a slice image of a portion of the organ.

According to some embodiments of the invention, the display calculation module is further operable to calculate a view based OCT scan data and on at least one of a group consisting of
a) information from a historical source; and
b) information from an imaging modality other than an OCT probe system.

According to some embodiments of the invention, the calculated view comprises calculated estimations of a non-observed position of a lesion, the estimation being based on observed portions of a presumed same lesion observed in data collected during a plurality of OCT probe penetrations.

According to some embodiments of the invention, the view is a stereoscopic view of a portion of the model.

According to some embodiments of the invention, the system further comprises an image analysis module operable to detect, in OCT scan data, a data pattern characteristic of an organ border.

According to some embodiments of the invention, the system further comprises an image analysis module operable to detect, on OCT scan data, a data pattern characteristic of a lesion.

According to some embodiments of the invention, the image analysis module communicates with a user upon detection of one of
a) an organ border; and
b) a suspected lesion.

According to an aspect of some embodiments of the present invention there is provided a method for creating a three dimensional map of at least a portion of an organ, comprising:
a) performing a plurality of insertions of at least one Optical Coherence Tomography (OCT) probe into tissue at a plurality of sites, each site differently positioned with respect to the organ;
b) using a processor to create a three-dimensional mapping of the at least a portion of the organ based on image data reported by the at least one probe during the plurality of insertions.

According to some embodiments of the invention, the method further comprises using a probe location module to report to the processor locations of the at least one probe during the imaging during the plurality of insertions.

According to some embodiments of the invention, the method further comprises using the processor to calculate, as a function of the imaging data and of information relation to position of the at least one probe during the imaging, position of an imaged feature in three-dimensional space.

According to some embodiments of the invention, the method further comprises using a same probe for sequential insertions;

According to some embodiments of the invention, the method further comprises using a plurality of probes for simultaneous insertions.

According to some embodiments of the invention, the method further comprises imaging an approximately cylindrical volume of tissue during each of the insertions.

According to some embodiments of the invention, at least some of the cylinders have overlapping portions.

According to some embodiments of the invention, the method further comprises performing the insertions in such a manner that the greatest distance between two adjacent cylinders at their most distant point is less than a pre-selected distance.

According to some embodiments of the invention, the pre-selected distance is the diameter of a tumor considered to be large enough to be considered clinically significant.

According to some embodiments of the invention, the method further comprises utilizing a second imaging modality in addition to the OCT probe to image the organ during insertion of the OCT probe in the organ.

According to some embodiments of the invention, the other imaging modality is an ultrasound.

According to some embodiments of the invention, the method further comprises using an ultrasound probe which comprises a guide for guiding insertion of a needling into tissue to guide insertion of the probe into the organ.

According to some embodiments of the invention, the method further comprises using only an OCT probe as an imaging device when inserting the probe into the organ.

According to some embodiments of the invention, the method further comprises using the processor to analyze image data from the probe to detect at least one of
a) imaging of a border of the organ; and
b) imaging of a lesion in the organ.

According to some embodiments of the invention, the method further comprises using a servomechanism to move the probe during at least some of the insertions.

According to some embodiments of the invention, the method further comprises
a) using a servomechanism to move the probe during at least some of the insertions; and
b) using the processor to calculate a command to the servomechanism subsequent to detection of one of
   i) an organ border; and
   ii) a tissue lesion.

According to an aspect of some embodiments of the present invention there is provided a method for 3D mapping of a region of interest in a body, comprising
a) using a probe-positioning module to insert at least one Optical Coherence Tomography (OCT) probe into a plurality of probe insertion sites within a region of interest in a body; and
b) using a 3D mapping module to calculate a 3D model of the region of interest, the calculation being based at least on part on
   i) a first data stream reporting position of the at least one probe at a plurality of positions during a plurality of insertions of the at least one probe in the region of interest; and
   ii) a second data stream generated by the at least one probe during the insertions and at the reported positions.

According to some embodiments of the invention, the method further comprises controlling positioning of the probe by the probe positioning module as a function of a detected characteristic of a tissue scanned by the probe.

According to some embodiments of the invention, the detected tissue characteristic is a detected organ border.

According to some embodiments of the invention, the detected tissue characteristic is a suspected tissue lesion.

According to some embodiments of the invention, the method further comprises controlling OCT probe insertions as a function of a characterization based on analysis of data from the at least one probe.

According to some embodiments of the invention, the method further comprises concentrated OCT scanning of a lesion detected during a less concentrated OCT scan.

According to some embodiments of the invention, the method further comprises directing an OCT probe penetration of tissue in a region of interest in a manner which avoids passing the OCT probe through the lesion, the directing of the probe being based on a calculation based on information about position of the lesion gleaned from another OCT probe insertion.

According to some embodiments of the invention, the method further comprises using OCT probe scanning to position a treatment probe with respect to a lesion detected by an OCT scan.

According to some embodiments of the invention, the treatment probe is a cryoprobe.

According to an aspect of some embodiments of the present invention there is provided a method for controlling insertions of an OCT probe into an organ, comprising:
a) inserting the OCT probe into an organ;
b) receiving image data from the probe during the insertion;
c) analyzing the image data to detect a characteristic of tissue being imaged by the probe; and
d) modifying movement of the inserted probe when a tissue is detected as having a predefined tissue characteristic.

According to some embodiments of the invention, the method further comprises
e) aiming a first OCT probe towards and into a body organ and scanning a portion of the organ during longitudinal movement of the inserted probe; and
f) ceasing forward motion of the inserted probe when a far border of the organ is detected by analysis of data from the inserted probe.

According to some embodiments of the invention, the method further comprises initiating an additional probe insertion at a predetermined lateral distance from a current probe insertion if a side border of the organ is not detected during a current insertion.

According to an aspect of some embodiments of the present invention there is provided a method for OCT scanning of an organ, comprising:
a) specifying a minimum diameter of a lesion defined as clinically significant by virtue of its size;
b) using at least one OCT probe to scan tissue of the organ during a plurality of probe insertions, and spacing the insertions so that a maximum distance between tissue volumes scanned during the plurality of insertions is less than the specified minimum diameter.

According to an aspect of some embodiments of the present invention there is provided a method for examining an organ over a period of time, comprising
a) performing a first OCT scanning of tissues of the organ, and creating a 3D mapping of the organ based on image data collected during a plurality of insertions of at least one OCT probe;
b) detecting location of a potentially dangerous lesion by analyzing results of the scanning;
c) performing, after a waiting period, a second OCT scanning of at least the detected location; and
d) comparing information based on image data collected during the first scan with image data collected during the second scan.

According to some embodiments of the invention, the method further comprises displaying a difference between data from the first scan and date from the second scan relating to the detected location.

According to some embodiments of the invention, the method further comprises displaying data from the first and second scans and highlighting detected differences on said display.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1A is a flowchart of an exemplary method for using an OCT scanning system, according to some embodiments of the present invention;

FIG. 1B is a simplified schematic showing action of an OCT probe scanning an organ or other region of interest, according to an embodiment of the present invention;

FIGS. 2A and 2B are respectively a side view and an end-on view of an organ showing exemplary schemes for achieving volumetric scanning coverage of the organ from sets of local images, according to some embodiments of the present invention;

FIG. 3 is a generalized view of an OCT scanning system using an ultrasound probe, according to some embodiments of the present invention;

FIGS. 4 and 5 are a general view and a more detailed view respectively of an OCT scanning system, according to some embodiments of the present invention;

FIG. 6 presents a simplified schematic of an OCT scanning system, according to some embodiments of the present invention;

FIG. 7 presents a simplified schematic of an OCT scanning system comprising a rectal ultrasound transducer, according to some embodiments of the present invention;

FIG. 8 presents a simplified schematic of an OCT scanning system which comprises a catheter-based OCT probe, according to some embodiments of the present invention; and

FIG. 9 presents a simplified schematic of an OCT scanning system which comprises a template, according to some embodiments of the present invention;

FIG. 10 is simplified schematic of a rotating OCT probe, according to some embodiments of the present invention;

FIGS. 11A-11C are views of an OCT probe which comprises a sharp tip attached directly to a rotating assembly, according to some embodiments of the present invention;

FIG. 11D is a simplified schematic showing an addition use for an OCT probe, according to an embodiment of the present invention;

FIGS. 11E and 11F, which are views from above and from the side respectively of an additional embodiment of an OCT probe which is also a biopsy needle, according to an embodiment of the present invention;

FIG. 12 is a simplified schematic of a miniature interferometer incorporated directly on an OCT probe, according to an embodiment of the present invention; and

FIG. 13 is a simplified schematic of an OCT probe which comprises a tiltable beam director, according to some embodiments of the present invention.

### DESCRIPTION OF SPECIFIC EMBODIMENTS OF THE INVENTION

The present invention, in some embodiments thereof, relates to tissue mapping and modeling systems and methods, and, more particularly, but not exclusively, to methods and systems for mapping and 3D model reconstruction of an organ, optionally in real time, using optical coherence tomography.

For simplicity of exposition, electromagnetic waves used by OCT probes will sometimes be referred to herein as “light”, but it is to be understood that wavelengths including visible light, Near-IR wavelengths and other IR wavelengths are also being referred to in references herein to “light” used in OCT probes.

An OCT probe module comprises a probe, optionally insertable in a body, and various light sources, sensors, motors, and optionally other equipment classically used to operate an OCT probe and to derive image data from the probe. As used herein, when appropriate according to context, the term “OCT probe” should be understood to include the probe itself and all other necessary parts of an OCT probe module required to operate it.

Typically, according to methods of prior art, only a relatively small volume of tissue is scanned, therefore OCT is useful for examining in detail a known lesion or known problematic anatomical structure. OCT techniques have not previously been used to scan a large volume or an entire organ for diagnostic purposes. Some embodiments of the present invention comprise means and methods for relatively large-scale diagnostic scanning of organs or parts of organs, and for mapping the scanned volume in a three-dimensional map and optional reconstructed model optionally displayed on screen and which enables comparisons with past and future OCT scans and with other forms of spatially specific diagnostic information, and which may serve as a guide to a therapeutic procedure.

Some embodiments of the present invention serve to overcome limitations of the range of OCT scanning. The current effective range of an OCT scanning operation in light-scattering tissue is only 2-3 mm, though this figure may increase somewhat as the technology develops.

OCT probes currently in use include ‘front looking’ and ‘side looking’ versions. Prior art methods of viewing comprise moving the scanning head (or a portion thereof) of an OCT scanner to send a light beam in a plurality of directions, for example by rotating a portion of a scanning probe, and thereby gleaning scan information from a plurality of directions or for example by moving an inserted probe longitudinally along a path of an insertion into tissue, and gleaning scanning information from a plurality of positions along that the pathway of that tissue insertion. Some embodiments of the present invention expand the scanning ability of OCT probe systems by providing means and methods for gleaning scan information from a plurality of OCT probes and/or from a plurality of tissue insertions of same OCT probe, recording that information in a common unified three-dimensional coordinate system, and thereby scanning and recording information from a tissue volume larger than that which can be scanned by a single probe in a single tissue insertion. OCT systems utilizing some embodiments of the present invention may be used to combine, coordinate, and collectively analyze information gleaned from OCT scans performed during a plurality of “tissue insertions” (insertion of OCT probe into tissue for scanning purposes). This plurality of tissue insertions may be performed by one probe in a plurality of sequential insertions, and/or by (optionally simultaneous) insertions of a plurality of probes into tissue. Both methods may be used to use OCT probes to scan a large tissue volume. In this manner, in some embodiments, an entire organ, such as for example a prostate, can be scanned in sufficient detail to detect clinically significant tumors or other lesions.

It is noted that scanning of an organ according to an embodiment of the invention may comprise insertions of probes into the organ and may also comprise insertions of probes into the body and around the organ. For example, an embodiment may comprise insertions into tissue near an organ and/or insertions (e.g. in a catheter) into a body lumen (e.g. a urethra) passing within an organ and/or insertions into a body lumen near an organ.

In some embodiments, a plurality of OCT probe insertions may be directed towards a vicinity of a previously detected lesion or suspected lesion, a lump in a breast for example, an may enable detailed and accurate mapping and optional 3D modeling and optionally pathological diagnosis of the suspected lesion. A detailed three-dimensional mapping and/or modeling of the lesion, optionally obtained from a plurality of OCT probe insertions into a lesion and/or into tissue around a lesion may provide a detailed guide for a surgical procedure. Alternatively, such a map and model may provide means for a series of detailed anatomical comparisons of views of a problematic region, taken over time.

The accuracy and detail of the scans made available by some embodiments of the present invention may in some cases provide a surgeon with treatment options which were not practical according to methods of prior art. For example, in the field of prostate surgery, discovery of a prostate cancer, for example through detection of an elevated PSA followed by a ‘shotgun’ core biopsy, generally results in a surgical decision to ablate the prostate, despite the fact that prostate ablation is known to produce deleterious side effects such as, incontinence, impotence, rectal problems and other types of collateral damage. According to methods of prior art, surgeons often opt for prostate ablation despite the fact that some prostate cancers are fast-growing and dangerous, while others are slow-growing and much less dangerous, because prior art fails to provide reliable and effective means for observing the behavior of individual tumors over time, at a resolution that enables timely intervention when a growth turns out to be dangerous. However, some embodiments of the present invention may enable alternative strategies, perhaps with better balancing of risk vs. benefits. For example “active surveillance” may become a treatment of choice for some detected prostate growths, because using some embodiments of the invention may in some cases enable “active surveillance” to be an exact and detailed and highly accurate observational process, as compared to the relatively blind and chancy process it had been according to methods of prior art. According to some embodiments of the invention, observation of a growth in a body tissue, such as for example a prostate, enables not only detailed observation of tissue structures in situ, but also detailed observation of growth or other changes in these tissue structures over time.

An important aspect of some embodiments of the invention is that they provide to a surgeon the possibility of mapping an entire organ or large portions of an organ, and the possibility of displaying 3D model of the organ on screen, and the possibility that the resultant mapping may be sufficiently large and sufficiently detailed to provide accurate and repeatable information relating the position, size, and shape of a lesion to known anatomical landmarks in the body, thereby making it possible to ‘register’ a scanning map based on a three-dimensional coordinate system with reference to known or scanned positions of known anatomical landmarks. Such registration of a scan mapping enables comparison of scan data from a plurality of scans performed over time.

Some embodiments of the invention may comprise one, some, or all of:

coordinating movement of a single OCT probe used repeatedly and/or a plurality of OCT probes, to effect a plurality of spatially coordinated penetrations of tissue in or near an organ or other region of interest;

during a scanning procedure, detecting positions of probes and of patient anatomy with respect to a three-dimensional coordinate system and reporting same in a ‘locations’ data stream. The locations data stream optionally includes information about positions of one or more imaging probes, and/or optionally includes information about movements of a region of a patient's body during scanning;

receiving data gleaned from a probe-based OCT imaging process in an ‘imaging’ data stream. The imaging data stream optionally includes information relating to distances and directions of imaged tissue features from imaging probes;

Calculating positions of imaged tissue features with reference to a three-dimensional coordinate system related or relatable to anatomic landmarks in a patient, the calculations optionally being based on information a locations data stream and from an imaging data stream. (It is noted that a locations data stream may comprise information about fixed or predictable positions of a probe and/or may comprise information based on sensor responses and/or reports from a probe positioning module);

Recording said features with reference to the calculated positions, the positions being identified in terms of a common three-dimensional coordinate system, thereby constructing a three-dimensional mapping (and optionally, display of a 3D model reconstruction) of an organ or other area of interest;

optionally detecting positions of one or more additional imaging modalities, and recording data gleaned from their operation also in terms of the common three-dimensional coordinate system;

analyzing data collected and mapped in the three-dimensional coordinate system to draw conclusions about tissues within the scanned volume and/or to monitor the scanning process;

recording data analyses and/or historical data (e.g. from a previous mapping of the organ or region of interest) and/or other known information about the organ or region of interest in context of a same three-dimensional coordinate system as that used to map scan data about the organ, thereby creating what is called herein an (optionally displayable) 3D model of the organ;

using a probe positioning module which comprises an automated servomechanism to move a probe used in the scanning process;

calculating commands to the servomechanism based on conclusions drawn from analysis of scan data;

planning and/or recommending probe placements according to pre-defined scanning criteria;

providing instructions and/or feedback to a user to facilitate his placing probes for scanning according to a plan;

providing instructions to an automated servomechanism, commanding probe placements and movements according to a scanning plan;

displaying scanned data from OCT scans and optionally from other imaging modalities in context of the common coordinate system;

displaying historical data in context of the unified coordinate system;

displaying a comparison of historical data with currently scanned data in context of the unified coordinate system, optionally highlighting differences; and/or

displaying data analyses in context of the unified coordinate system.

For simplicity of exposition, electromagnetic waves used by OCT probes will be referred to herein as “light”, but it is to be understood that wavelengths including visible light, Near-IR wavelengths and other IR wavelengths are also being referred to in references herein to “light” used in OCT probes.

Before explaining at least one embodiment of the invention in detail, it is to be understood that the invention is not necessarily limited in its application to the details of construction and the arrangement of the components and/or methods set forth in the following description and/or illustrated in the drawings and/or the examples. The invention is capable of other embodiments or of being practiced or carried out in various ways.

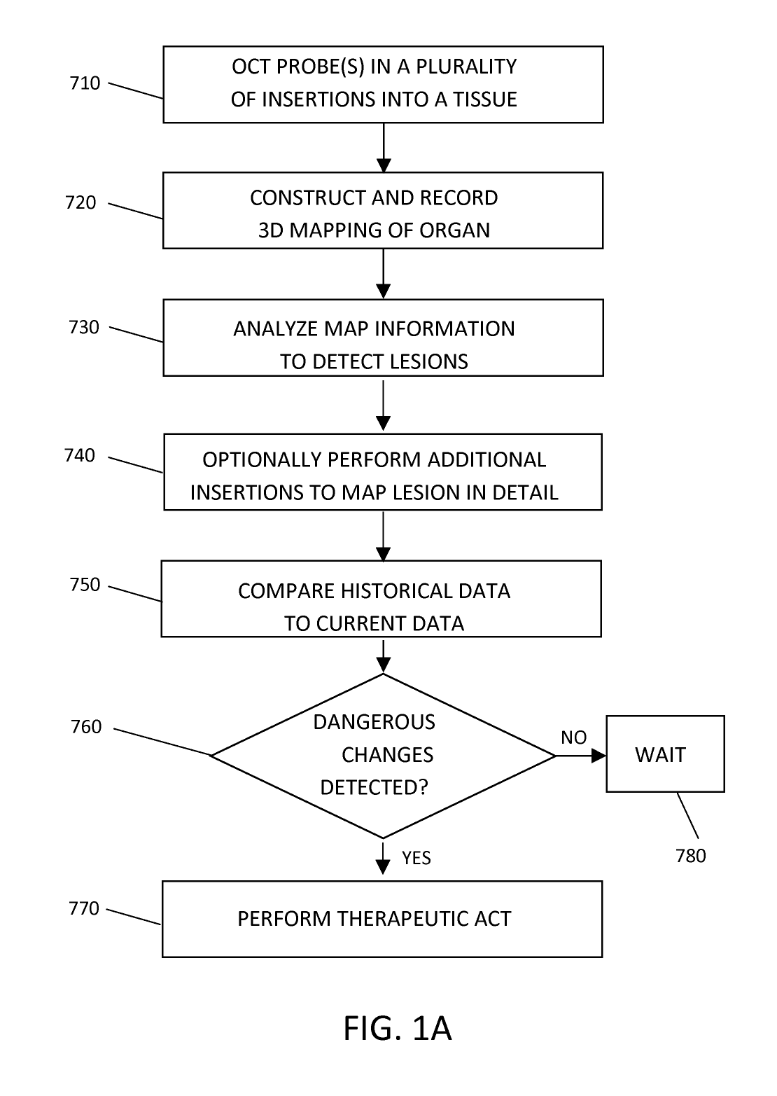

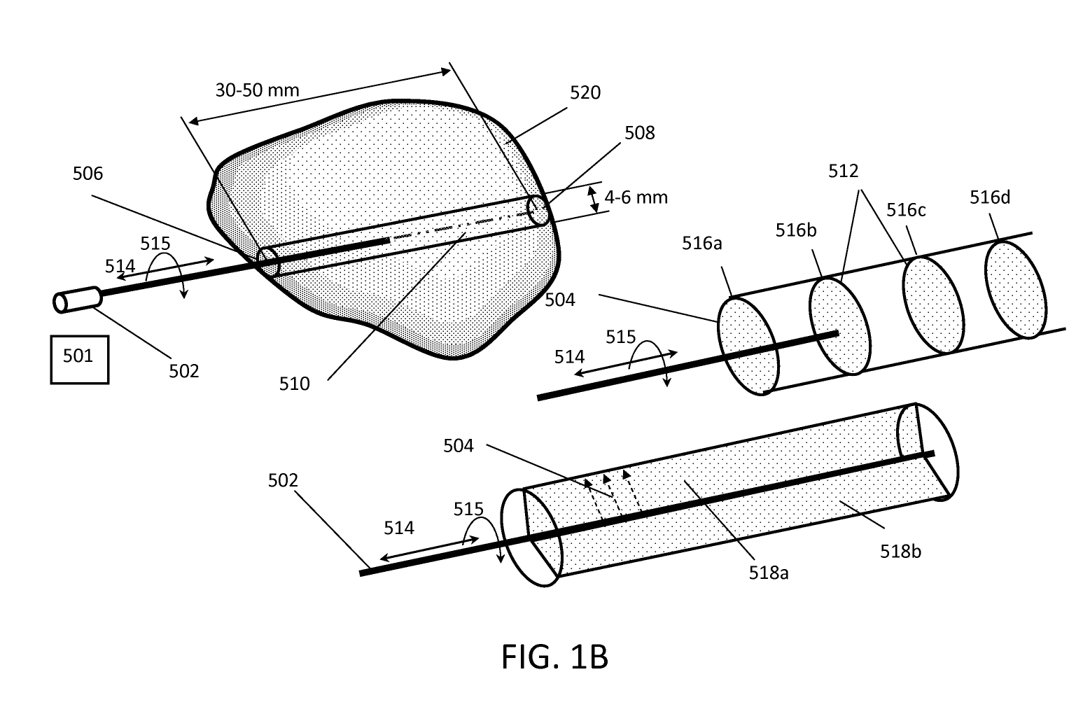

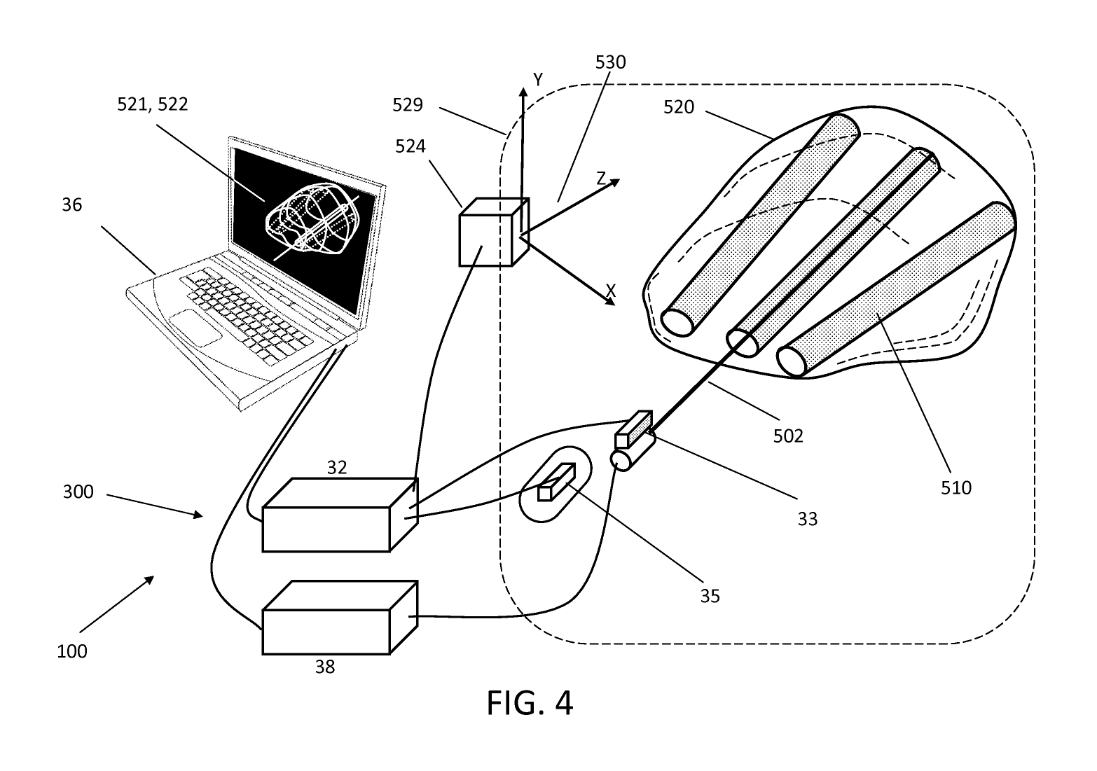

Referring now to the drawings, FIG. 1A is a flowchart of an exemplary method for using an OCT scanning system 100 (shown in FIG. 4) according to some embodiments of the present invention in an “active surveillance” procedure for handling suspected tumors in an organ such as a prostate. The method comprises:

(710) performing a plurality of OCT probe insertions into tissues (optionally into an organ and/or into tissue near an organ and/or into a body lumen near an organ), operating the scanning probes while at least partially rotating them and/or advancing and retracting them, to create imaging data in a plurality of directions from a plurality of positions;

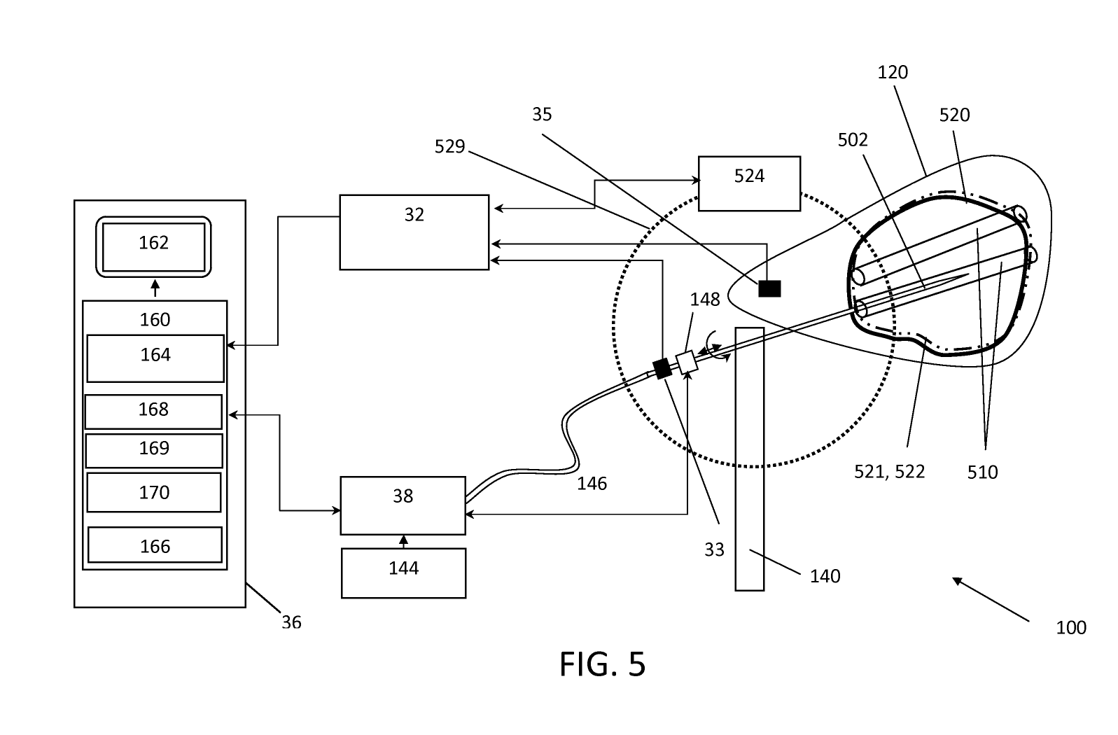

(720) constructing and recording a 3D mapping and modeling of at least a portion of the organ, the mapping comprising information gleaned from operation of one or more OCT probes during a plurality of OCT probe insertions. The mapping procedure optionally makes use of image data generated by the OCT probe module and/or of probe location data generated by a location sensor module and/or a probe positioning module and/or sensor information reporting movement of the organ being scanned and/or imaging information from additional (non-OCT) imaging modalities. The mapping is recorded by relating received image data to its calculated point of reference in the tissue. In other words, optionally, information about the position of the OCT probe (dynamically generated or known to the system) and optionally information about the position of the scanned organ or tissue is/are used to calculate the position with respect to a three-dimensional coordinate system of objects and features observable in the scanned image data. In some embodiments a unified coordinate system is used, and positions of patient, of surgical tools including OCT probes, of surrounding anatomy visualized by additional imaging modalities such as ultrasound, CT, fluoroscope, and MRI, and/or of OCT-scanned features may all be expressed and optionally recorded in terms of that unified coordinate system and optionally modeled and displayed. Alternatively, a plurality of coordinate systems may be used, and a processor programmed to relate one coordinate system to another. For example, for convenience, a tool-locating module using a sensor attached to or embedded in an OCT probe and which is operable to report its own position may be used. In some embodiments the sensor may be sensitive to an electric field or radio signal broadcast, as shown for example in an exemplary embodiment shown inter alia in FIGS. 4 and 5. Alternatively, the sensor may use optical or electromechanical or combined techniques, receive and interpret an electromagnetic or optical or other signal produced by a probe, or may use any other technology to detect and report location of the probe. In some embodiments, mapping of an organ may be defined with reference to a coordinate system related to anatomical landmarks of a patient, landmarks which do not change from one scanning session to another. For simplicity, discussions herein refer to a unified coordinate system under the assumption that when it is necessary to translate information from one coordinate system to another, for example to relate coordinates of a probe-locating system in a room to coordinates defined with reference to anatomical landmarks of a patient, a processor may be operated to translate from one coordinate system to another. Optionally, the mapping may be displayed on a display, e.g. in slice, perspective, stereoscopic, and/or any other format.

(730) Optionally, mapped information may be analyzed to detect suspected lesions, for example tumors. This analysis may be manual, that is, may be performed by a surgeon or other medical practitioner. Alternatively or additionally, the analysis may also be performed by a processor running an image analysis algorithm programmed to recognize, in image data, features known to be associated with problematic tissue. Optionally, these analyses may be performed in real time, so that the results are available to the practitioner performing the scanning. Optionally, results of the analysis may be displayed on the display, for example in the form of highlighting, or in the form of a display of a hypothesized lesion whose position in non-scanned tissue is inferred from tissue characteristics observed in scanned tissue.

(740) Optionally, detection of a lesion in scanned tissue and/or inference of the presence of a lesion in non-scanned tissue may invoke (manually or as result of an animated process) additional probe insertions to better observe region detected to be problematic. Detection of a problematic region may happen in real time during a scan, or may be recorded in historical data, for example data and/or analyses recorded during a previous scan.

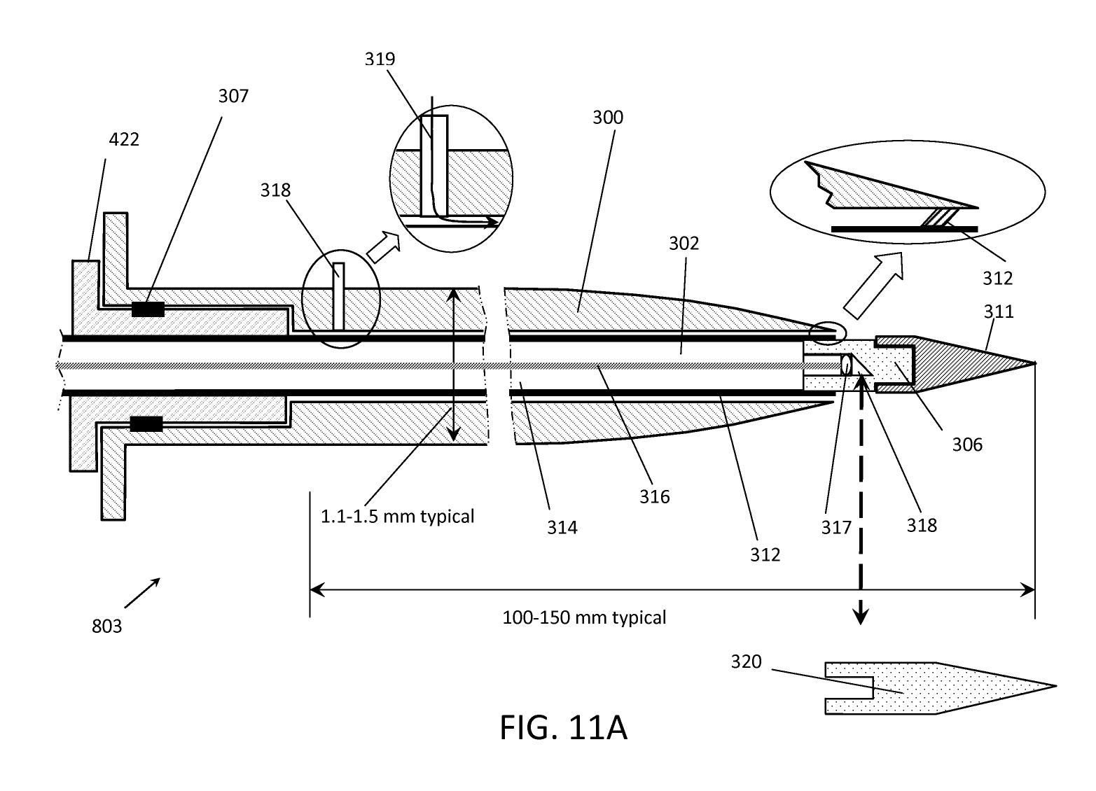

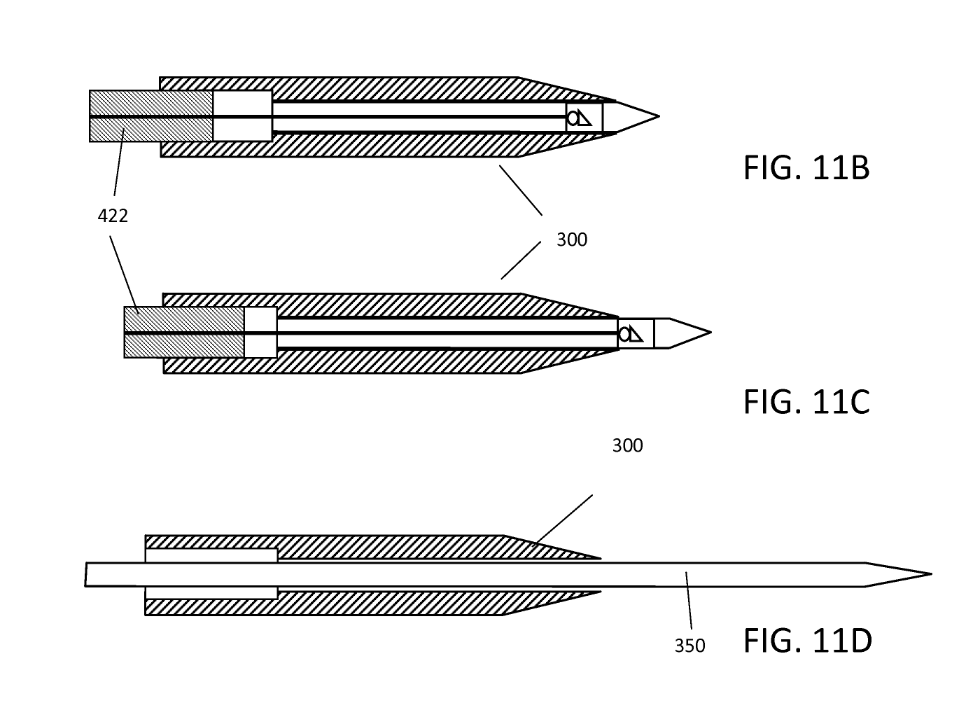

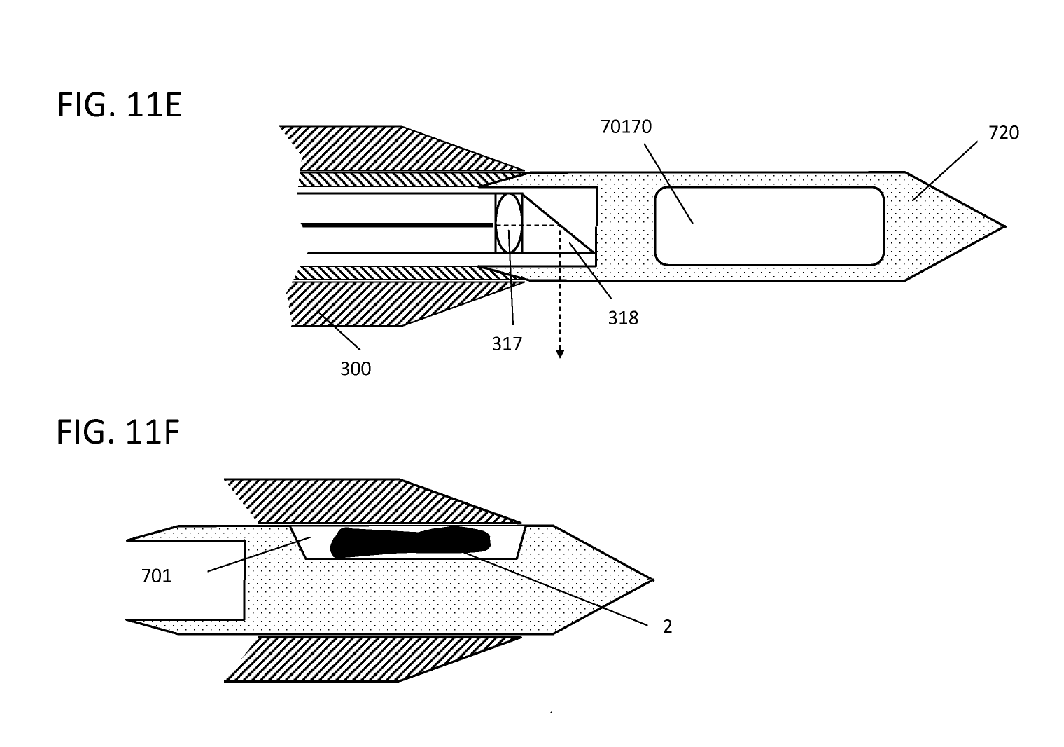

In an optional embodiment, a probe such as that disclosed in FIG. 11E may be used to take biopsy sample of the problematic tissue.

(750) In some embodiments, a scan as described above may be repeated, or information from non-OCT historical scans may be used. In either case, optionally, historical scan data may be related to real-time scan data by organizing both historical and real-time data with respect to a unified coordinate system. A display may then be used to compare old and new data, and automatically generated and/or practitioner-marked highlighting may be used to aid in comparing old and new data and in identifying and evaluating observable changes.

Evaluation of observable changes by a medical practitioner and/or algorithmic analysis may detect a change thought to be dangerous. A practitioner, optionally guided by algorithmically generated recommendations from scanning system 100, may decide (760) to perform a therapeutic act (770) such as ablation of what he perceives to be a dangerous tumor.

Alternatively, if no dangerous changes are detected, a practitioner may decide on a waiting period (780), followed by a follow-up scan (710).

Attention is now drawn to FIG. 1B, which is a simplified schematic showing action of an OCT probe scanning an organ or other region of interest, according to an embodiment of the present invention. An OCT probe 502 is shown penetrating from one side to the other of an organ 520. Probe module 501, optionally comprising console, light source, electronics, motors, communication equipment and/or other tools and components required for functioning of probe 502, is also shown. Optionally, probe module 501 may be attached to or contained within the body of probe 502.

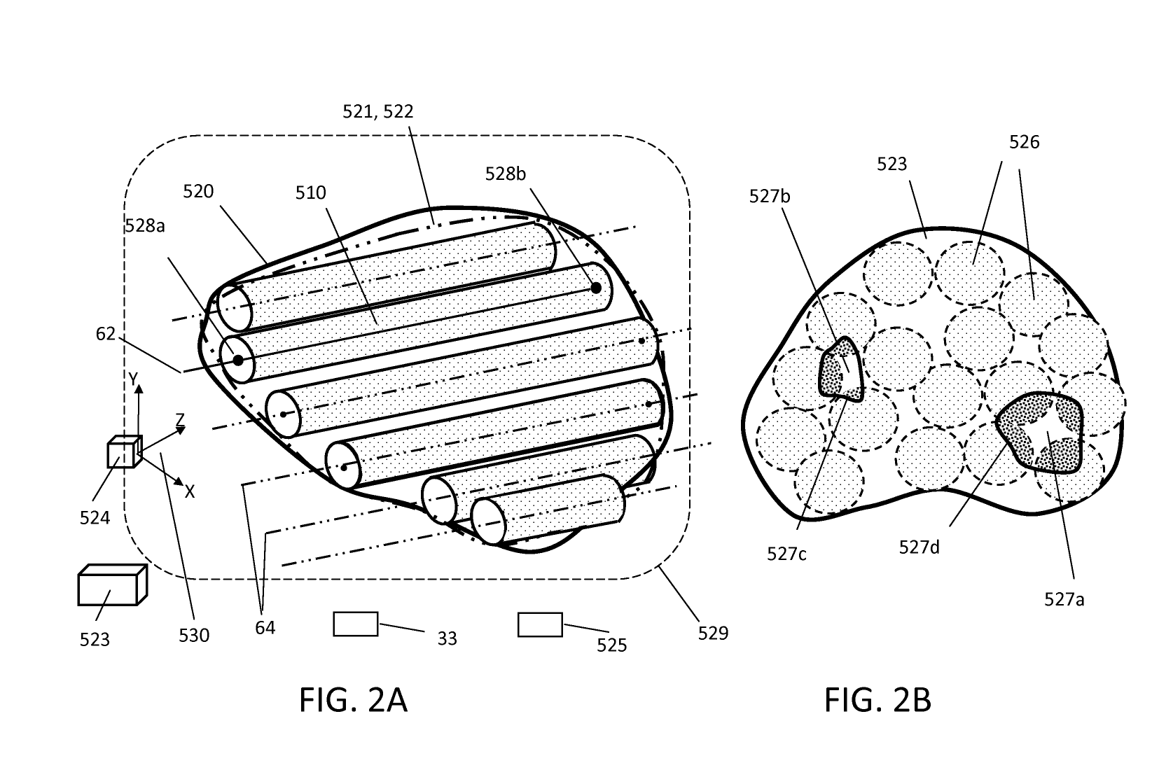

Prior to discussing in detail some OCT probes and some uses thereof, it should be understood that discussions herein of OCT scanning systems and methods, except insofar as these relate to exemplary probes according to some embodiments of the invention, are not to be considered limiting. With reference to FIGS. 1B, 2A, 2B, and various other figures, typical and popular types of OCT probes are described. These are exemplary probes and not to be considered limiting. Probes useable for optical coherence tomography and having structures and methods of operation different from those described herein may yet fulfill the roles describe for “probe 502” and other probes discussed herein. Specific arrangements of components and methods of scanning as described, for example, with reference to FIG. 1B should be understood as being exemplary and explanatory, but not as limiting aspects of the invention relating to a multi-insertion scanning system and various other embodiments described herein which in themselves are not dependent on particular probe structures and/or methods of use.

In some well-known structures and uses of an OCT probe, a probe such as probe 502 performs successive rapid axial measurements while scanning transversely around the probe, for example by rotating the probe or a component thereof as shown by arrow 515. This process, along with appropriate support activities of the probe module 501 as described above, may produce a two-dimensional data set that represents scanned image data from a cross-sectional plane through the tissue. Image data so gleaned can optionally be presented as a two-dimensional ‘slice’ transverse to the direction of insertion of the probe image showing microstructures of the body tissue. Such slices are shown as 516a, 516b, 516c, and 516d. Diameter of such a slice will typically be between 4 mm and 6 mm using today's OCT technology, although diameters larger and smaller are possible, depending on resolution desired and on opacity and density of a particular tissue.

In a distinct but similar process, scanning laterally in a direction while advancing or retracting a probe through tissue longitudinally (i.e. in the direction of penetration of the probe, or the opposite) can produce a 2D data set in another dimension, a narrow flat longitudinal slice. In the figure, scanning in direction 504 produces image data in plane (and producing imaged rectangle) 518a. Scanning in a second direction while advancing/retracting probe 502 produces image data from plane 518b.

Combining both process, rotating the probe and also advancing and retracting the probe, can image tissue within an approximately cylindrical volume, with resolution of the imaged planes depending on speed of response of the probe and speed of movement of the moving components. Therefore, in some uses of an OCT probe, combining rotating of a probe with advancing and/or retracting the probe while sending out light beams and interrogating them using interferometery can produce a three dimensional data set describing some or all tissues within range of the probe. Although width of the cylinder is typically in the 4-6 mm range, length of the cylinder is potentially as long as the length of the probe penetration into tissue, or as long as the length of the probe penetration in the imaged organ, whichever is considered desirable.

OCT probes are typically thin, 0.5-3 mm. Internal structures of the optics requires only a core a few microns in diameter, while the clad outer diameter may be few hundred microns. These thin probes can be used to penetrate and scan prostate, breast, liver, and a variety of other tissues doing minimal damage and with minimal pain (though some patients will want local sedation).

FIG. 1B shows an exemplary embodiment where an OCT probe 502 is used to scan a portion of a prostate 520. OCT scanned volume 510, within organ 520, includes and surrounds the insertion path of probe 502. In scanning the prostate, for example, probe 502 may be inserted into the prostate gland from its apex and up to the bladder. In typical use, as known in the art, a user sees in real time an image of the scanned volume and can specifically observe and record the ‘near’ and the ‘far’ borders of the organ. Length of probe penetration in scanning a prostate would typically be 30-50 mm, as shown in the figure.

Assuming OCT probe 502 is a probe with a side view (examples of which are discussed below), meaningful OCT data can be gathered to a depth of depth of 2-3 mm from the probe, consequently during a single penetration of probe 502 in organ 520, meaningful image data may be collected from a cylinder 30-50 mm in length and 4-6 mm diameter. It may be noted that the volume of a standard core biopsy from the prostate has a mean length of 12 mm and mean diameter of 0.4 mm, therefore OCT image data from a single penetration provides detailed information on the microstructures of an amount of prostate tissue approximately 520 times larger than that produced by a standard biopsy sample. This difference is significant not only because any given OCT probe penetration and imaging is far more likely to discover an existing pathological condition than is a comparable penetration by a biopsy needle, but also because the percentage of organ 520 observable during a single penetration is such that it is practical to contemplate detailed scanning of an entire organ. The planning and organization of such scans are discussed in the following.

Regardless of the probe movement strategy used during the penetration (longitudinal scan, rotational scan, combination of both, helical, other . . . ), in some embodiments the instantaneous position of probe 502 may be reported to the system at the same time or nearly the same time as imaging date is being reported. A probe location sensor 33 for this purpose is shown in FIG. 4, and other location-reporting options are discussed below.

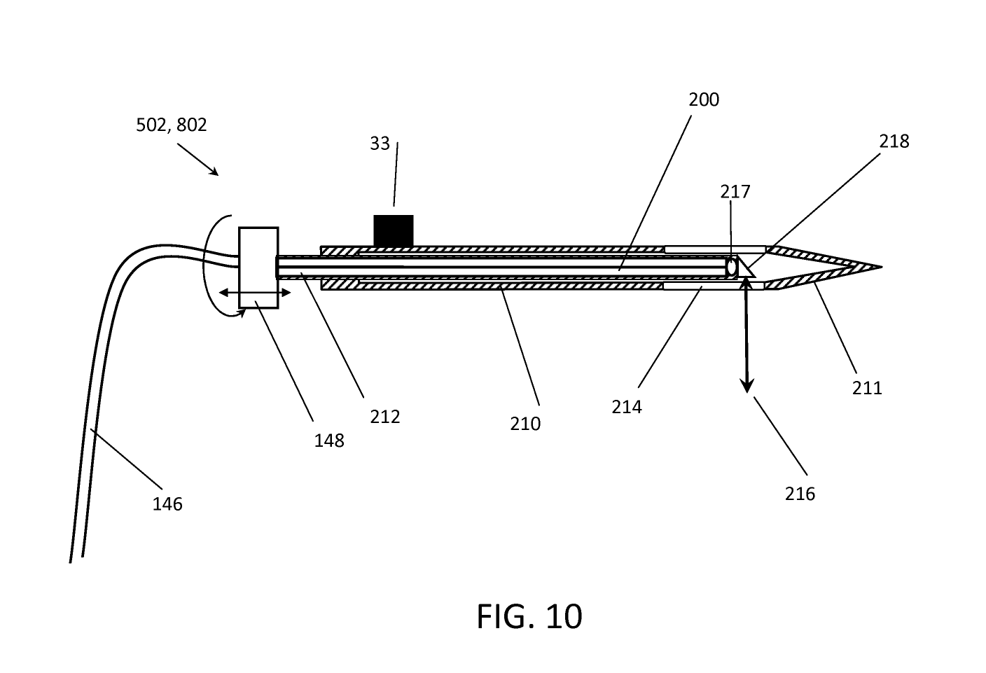

It is noted that the scanning techniques discussed above and shown in FIG. 1B are exemplary only, and not to be considered limiting. Other scanning techniques may be used, for example various ways of combining translation and rotation modes in using side-viewing OCT probes, and front-viewing OCT probes or other types of OCT probes may be used also. A detailed example of a side-viewing OCT probe is presented in FIG. 10 and discussed below. A front-viewing OCT probe, such as, for example, the NIRIS system sold by Imalux Corp. of Cleveland Ohio, U.S.A., and currently viewable at www.imalux.com, can be used as well.

Attention is now drawn to FIG. 2A and FIG. 2B, which are respectively a side view and an end-on view of an organ 520, showing exemplary schemes for achieving volumetric scanning coverage of organ 520 from sets of local images, according to some embodiments of the present invention.

In some embodiments, image data collected during a plurality of OCT probe penetrations of an organ are associated with positions in a three-dimensional coordinate system 530. Calculations based on data from one or more probe modules 501 reporting position of imaged tissue features with respect to an imaging probe 502, together with data from a location tracking system 32 (see also FIG. 4), represented here also with its field generator 524 receiving data from a location sensor 33 attached to or incorporated in probe 502 (or from other probe location information sources, as discussed below) enables tracking system 33 to calculate the location of an imaged feature with respect to a three-dimensional mapping 522 and 3D modeling 521 based on common three-dimensional coordinate system 530 and thereby related to real positions of things in the operating environment and/or related to positions of landmarks of a patient's anatomy.

Each cylinder in FIG. 2A represents a volume from which imaging data has been gathered by a single penetration of an OCT probe 502. Optionally, a single probe used for repeated penetrations of organ 520 may gather this data, one ‘cylinder’ per penetration. Alternatively, penetrations by a plurality of probes 502, operating sequentially and/or simultaneously, may gather this data.

(It is to be understood that scanned volumes are not necessarily of cylindrical shape. As shown in FIG. 1B, for example, a scan may cover only a part of a cylinder, for example a pie-slice portion of a cylinder, or a simple plane, or for that matter any arbitrary (random or planned) shape. Indeed, in the case, for example, of a curved OCT probe, a scan penetration path might optionally have no straight component at all.)

Within each cylindrical local volume 510, imaged data are scanned and recorded at high resolution, with resolutions on the order of 1-10 microns. Each reported data point therefore may carry OCT-generated information and may also be identified with respect to its spatial location within coordinate system 530. Depending on degree of overlap among scanned volumes 510, imaged data recorded as being in positions identified with respect to coordinate system 530 may constitute a full or a partial filled data picture organ 520.

According to some current medical practices, only tumors larger than pre-selected size are considered to be “clinically significant”. In current practice regarding prostate tumors, those larger than 0.5 cubic centimeters or about 10 mm in diameter are considered clinically significant by some physicians. Therefore, in some embodiments, probe insertions are planned to only partially fill organ 520, with no overlap, so as to use a minimum number of insertions (to reduce pain, an possible infections, and to save time) while still being assured that all tumors whose diameter is large enough to be considered clinically significant will be imaged, at least in part. Moreover, since borders of a small tumor which happens to fall mainly between imaged ‘cylinders’ will appear in at least some and possibly all of the surrounding cylinders, partial imaging of the tumors can in some cases suffice for a qualitative and partially quantitative understanding of the location, size, and shape of such tumors.

Such a situation is shown in FIG. 2B, where two exemplary small tumors, 527a and 527b, are shown in positions where the center of the tumor is situated in non-imaged tissue. The figures show that given scanning coverage as shown in the figure, only the narrowest tumors can escape being imaged at all. In cases 527a and 527b, imaged portions of the tumors suffice not only for detection of the tumor but also as a basis for some reasonably accurate guesswork as to the size and position of portions of the tumor in non-imaged tissue. It is noted that ‘guesses’ (i.e. estimates) of this sort may in some embodiments be calculated by an analysis module and results of the analysis may be displayed on a display. Optionally, a display of the scanned data and/or a display of data stored in the three-dimensional mapping may include highlighted detected abnormal tissue and/or estimates of possible tumor presence in locations of non-imaged tissue.

It is noted also that the scanning distribution schemes shown in FIGS. 2A and 2B are exemplary only, and not limiting. In general, a medical practitioner using the system may choose a scanning density according to his appreciation of the medical requirements of the case, and optionally in some embodiments a planning and recommender module 523 may recommend a density based on known characteristics of the case and known recommended medical practice for cases of that character.

Optionally, in some embodiments a system planning and recommender module 523 may optionally specify locations for probe insertions which will produce scans at the required density. For example, in a tissue expected to have slow-growing low-grade cancers or benign growths, a spare array for used for a periodic scan may suffice, (and may be preferred, since it is less painful and less time consuming) while a tissue suspected of harboring fast growing and dangerously malignant tumors, dense scanning which leaves no non-imaged tissue between ‘cylinders’ may be used.

In some embodiments, planning and recommender module 523 passes its recommendation for probe insertion locations to an automated probe positioning module 140 (see FIG. 5) which inserts probes at the recommended locations. In some embodiments those recommendations may be passed on to a practitioner who executes them, optionally with help from a probe placement assistance module which provides feedback and/or instructions to a user, to help him to manually insert a probe at a desired position and orientation and for a desired distance, according to the user's request and/or according to a recommendation from planning and recommender module 523.

In some embodiments, a user first manually inserts a probe to execute a first penetration, and thereafter recommender and planner 523 optionally computes a recommended insertion path for a next insertion as a function of the detected position of the manual scan (the scanned data being optionally registered in 3D map 521). For example, in FIG. 2B, insertion 62 might be a first (e.g. manual) insertion, and insertions 64 might be subsequent insertions recommended by recommender 523.

It is also noted that in the case of detection of a tumor or suspected tumor or other lesion or anomaly, in some embodiments planning and recommender module 523 may recommend, or a physician may on his initiative request, an additional scan an additional scan in which additional penetrations aimed in view of the detected problematic tissue site are performed. Such an additional scan may be performed by an automated system or performed manually. A user might wish or the recommender might recommend additional tissue insertions at or near the problem area, optionally from a different direction than the original scan, optionally providing overlapping ‘cylinders’, so as to provide more detailed information about the problem area. Optionally, using tumor location information already available in the 3D mapping at this point, addition probe insertions may be carefully aimed so as to approach but not touch a problem area, thus avoiding an interaction which might provoke a metastatic event.

### PROBE SYSTEM USING ULTRASOUND

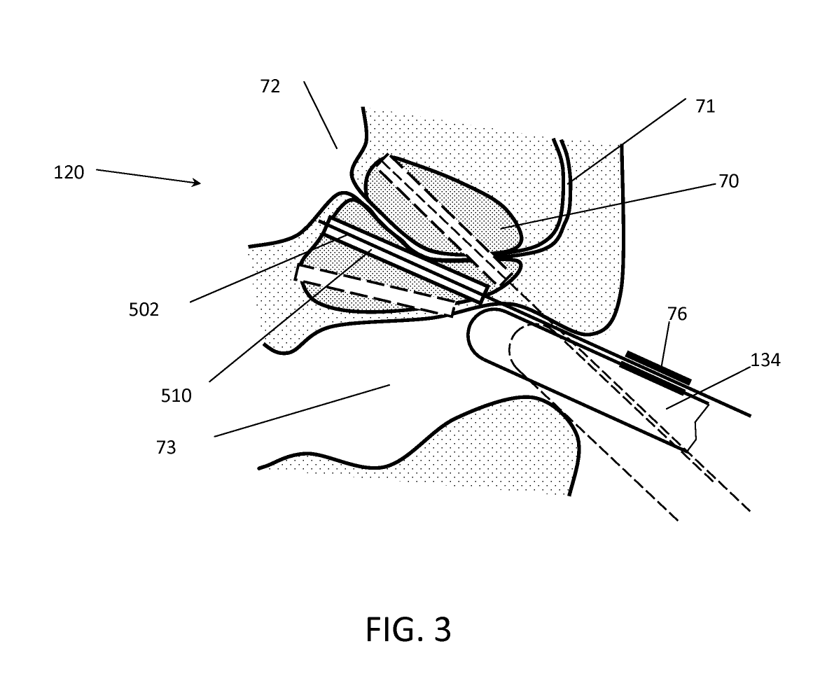

Attention is now drawn to FIG. 3, which presents a generalized view of an OCT scanning system using an ultrasound probe, according to some embodiments of the present invention.

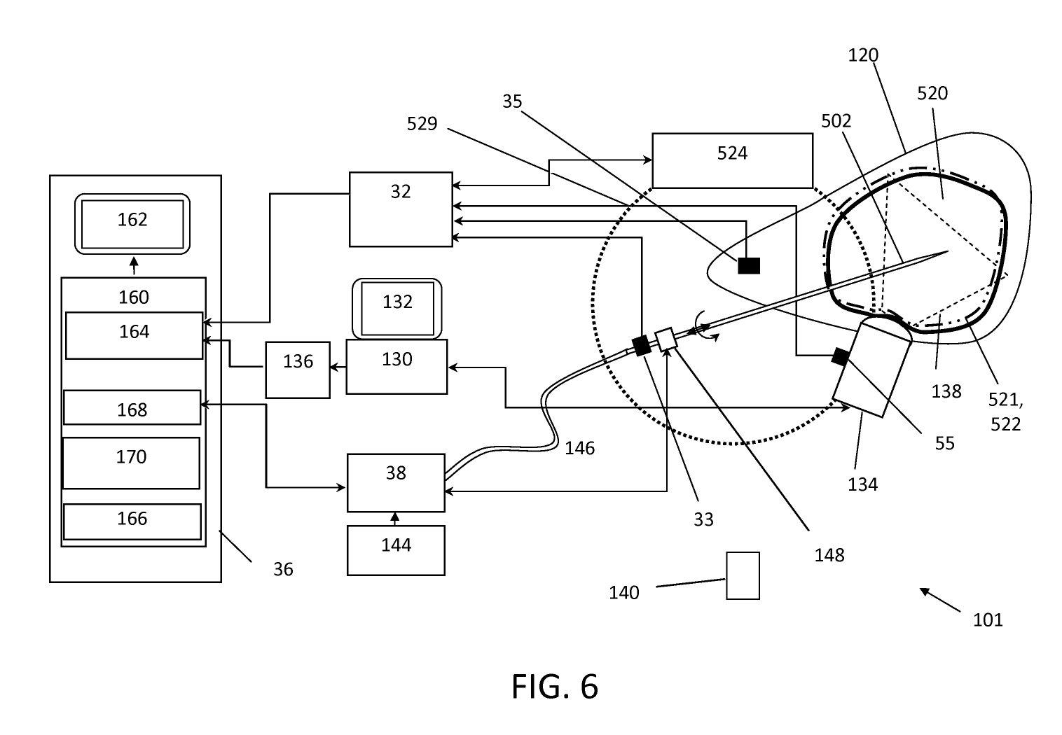

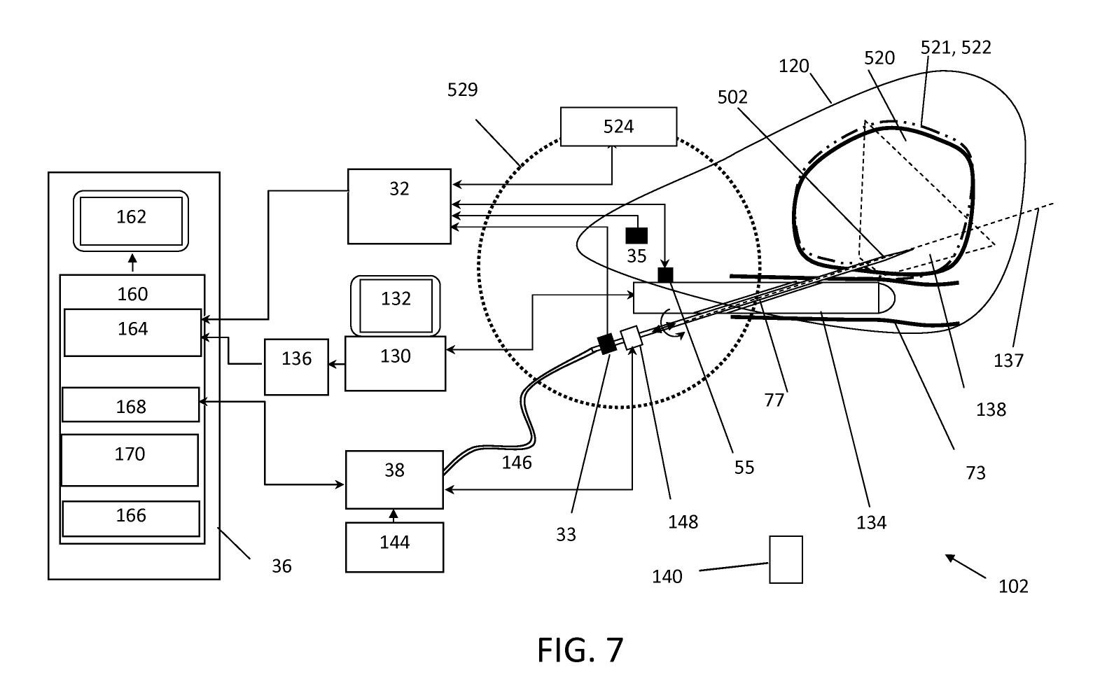

FIG. 3 shows an optional method for inserting OCT probes into a prostate, using an ultrasound probe to guide a plurality of insertions. In the figure patient 120 is undergoing a multi-insertion OCT scan using transrectal ultrasound-assisted OCT insertion. Shown in the figure are prostate 70, urethra 71, bladder 72, and rectum 73. According to some embodiments, an OCT probe is inserted into a prostate via a needle guide 76 comprised in or attached to an ultrasound probe (transducer) 134, in a manner made familiar by classical ultrasound-guided ‘core’ needle biopsies of the prostate. An OCT probe 502, in a form of a needle, is inserted through an external needle guide 76, or through a cannula needle guide 77 (depending on transducer model, see FIG. 7). The guides physically guide (limit the direction of) the inserted needle, while the ultrasound image shows a user where his needle is and/or what it is pointing towards. As shown in the figure, an OCT probe 502 is guided into a prostate 70 which is being imaged by ultrasound scanner 130 (shown in FIG. 6). In this exemplary embodiment probe 502 is caused to penetrate through the length of the prostate and up to the prostate border near bladder 72. Each insertion of probe 502 scans an approximately cylindrical volume 510 around the penetration path.

Ultrasound scanner 130 enables a user to insert his probe 502 in a manner which he considers desirable in view of an image of the organ appearing on the ultrasound display screen 132, for example a user may use the ultrasound display to achieve an even distributions of probes into a plurality of insertion paths in and/or near an organ.

Attention is now drawn to FIGS. 4 and FIG. 5, present a general view and a more detailed view respectively of an OCT scanning system 100 according to some embodiments of the present invention. A locator module 32 produces a location data stream 164 (shown in FIG. 5) and an OCT module produces an image data stream 168, both reporting to a central processor 160. Optionally, processor 160 calculates positions of features of the imaged tissue by combining information on the positions of imaged features with respect to 502 with information about where probe 502 was positioned when doing the imaging.

System 100 may comprise some or all of the following components:

An optical coherence tomography console 38, and one or more OCT probes 502.

The OCT probe 502, optionally with diameter of 0.25-5 mm, and a length optionally between 10 cm and 40 cm, and optionally having a tissue depth penetration capability of 1-5 mm, optionally has a shape of a needle with a sharp distal head. Probe 502 optionally comprises a transparent window for transferring light signals in and out of the probe. Such probes are usually sealed all around to prevent penetration of materials from the body into the probe upon insertion of the probe into the body.

Examples of an OCT probe that can be used as probe 502 are taught in U.S. Pat. No. 6,564,085 to Pitris et al., and U.S. Pat. No. 7,952,718 to Xingde Li et al. As another example of a probe 502, a probe according to some embodiments of the present invention, is discussed below.

OCT Console 38, include hardware and software optionally for organizing and communicating image data stream 168, that is, transferring OCT-generated diagnostic information to processor 160 where it may optionally be used for real time display, storage in a memory, interpretation and analyses, comparison with historical data, and/or 3D mapping.

An optional image analyzer 169 (shown in FIG. 5) optionally analyzes information contained in image data stream 168 and, for example using known techniques of pattern recognition, may recognize features of imaged tissue. In particular, analyzer 169 may recognize an organ border and report, for example, ‘entrance’ point 528a and ‘exit’ point 528b, both shown in FIG. 2B. Analyzer 169 may also make pathological analyses, reporting, for example, tissue suspected of being cancerous. (Data analysis modules for making such analyses are know in the art.)

Spatial Positioning Tracking (localization) System 300:

A second data stream, location data stream 164 (shown in FIG. 5), may optionally be generated by a location tracking module 300. In the exemplary embodiment of FIG. 4, location tracking module 300 comprises an electromagnetic field generator 524 which produces an electromagnetic field throughout a volume 529, a volume large enough to include at least part of the body of a patient and all the electromagnetic location sensors. Location tracking module 300 further optionally comprises a probe location sensor 33, optionally a 5 or 6 degrees of freedom sensor) mounted on a probe 502, and further optionally comprises a body location sensor 35 mounted on a body of patient 120, optionally, for example, on the L5 vertebra, whose movements have been found to correlate with movements of the prostate. Sensors 33 and 35 can detect and report their own positions and orientations as a function of detected electromagnetic field or other signals generated by field generator 524. Sensors 33 and 35 can have a wired or wireless connection to an optional location console 32 which optionally collects, interprets, digitizes, and/or communicates data from sensors 33 and 35 to central processor 160.

The exemplary embodiment described in the preceding paragraph is exemplary and not limiting. Other embodiments of location tracking module 300 are contemplated. For example, some embodiments utilize a probe positioning module 140 (shown in FIG. 5) useable to position a probe 502 at a desired position and orientation. A positioning module 140, for example, might be operable to report location of a probe 502 as it is moving, without need of sensors (e.g. by reporting location based on commands sent to a stepper motor). In some embodiments, templates or other forms of probe guides may be used to constrain movements of probes. In such a case, probe location tracking may be highly simplified or unnecessary, since probe location information might then be known in advance and available to processor 160 for calculations.

Examples of commercial systems that could serve as location tracking module 300 include electromagnetic tracking (e.g. Ascension Technology corp. Burlington, Vt., USA, and NDI's Aurora tracking system, Waterloo, Ontario, Canada), electromechanical tracking (cf Eigen LLC., Calif., USA , Biobot Pte Ltd., Singapore), optical tracking (e.g. NDI, Polaris tracking system, Waterloo, Ontario, Canada), IR tracking, 4D Ultrasound tracking(e.g. GE Ultrasound, USA, Koelis, La Tronche, France), gyroscopic tracking (U.S. Pat. No. 6,315,724), and accelerometers tracking (e.g. SENSR, Elkader, Iowa, USA, GP1 3 axis accelerometer and Gecko accelerometer).

Processor 160 receives probe location data stream 164 (optionally comprising real time information about locations of probes in real space and location of a body of patient 120 in real space), and also receives image data stream 168, optionally constituting actual image data from probe 502 and/or probe module 501. In other words, processor 160 receives information about what probe 502 is imaging and where it was imaging it from. Combining information from these two sources (optionally in real time) produces information about the position of imaged objects (e.g. tissue features) with respect to a three-dimensional coordinate system. A collection of this data is referred to herein as 3D mapping 522. A combination of mapping 522 with other spatially distributed information, for example with historical information from a previous scanning operation of a same tissue, is termed 3D model 521. Model 521 is optionally displayable according to a variety of views.

In some embodiments, using simultaneously the OCT device 38, and location tracking module 300, each OCT data point is further registered spatially by the tracking system, console 32, and transmitter 524, and sensor 33, mounted on the OCT probe. Sensor 35 is mounted on the patient body in order to monitor its instantaneous movements and to compensate for such movements, relating the whole set of data to one position of the patient within the transmitter coordinate system. Using an optional six degrees of freedom data from the sensors 33 and 35, standard vector calculus may be used for calculations for compensation for patient movements in calculating each data point.

Main Computer Control and Display:

In an exemplary embodiment shown in FIG. 4, a computer and display 36 provides processor 160, optional display 162, optional user interface 170 and an optional data storage unit (not shown). Optional OCT console 38 (shown in FIG. 5) and optional location module 300, optionally connect to processor 160 for transmitting and optionally for receiving data.

Probe Positioning Module 140

Some embodiments comprise a probe positioning module 140. Positioning module 140 is optionally a servomechanism commandable by commands sent from processor 160 and serving to physically position a probe 502 at a desired position. In particular, module 140 may be used to insert one or a plurality of probes 502 at pre-planned positions in organ 520, as discussed inter alia with reference to FIGS. 2A and 2B.

Display 162 optionally displays views of 3D mapping 522 and 3D model 521, temporal and spatial location (position and orientation) of probe 502, historical data from model 521 together with real time data from probe 502 and/or mapped data from mapping 522, and/or optionally coordinated data from another imaging modality such as an ultrasound probe. User interface 170 optionally comprises screen tools for manipulating the display, behaviors of various parts of the system, operational parameters, and various other instructions to the system. Interface 170 also optionally provides probe placement instructions and/or feedback to a user using system-guided manual placement.

Probe actuator 148 is a component of probe module 501, and is responsible for imparting to a component of probe 502 the longitudinal (514) and rotational (515) movements required for scanning.

Attention is now drawn to FIG. 6, which presents an OCT scanning system 101 according to some embodiments of the present invention. System 101 differs from system 100 in that it further comprises an ultrasound scanner comprising transducer 134, US console 130, and US display 132. Transducer 134 optionally comprises a sensor 55 operable to report position and orientation of transducer 134 to location tracking module 300. An optional ultrasound interpreter 136 (optionally a frame grabber) is operable to transfer a data stream from the ultrasound system to processor 160, which may optionally integrate this ultrasound-based imaging (or other US-based data) with OCT imagine and/or display of mapping 522 and/or display of model 521. Ultrasound transducer 134 is an abdominal transducer in this embodiment.

Attention is now drawn to FIG. 7, which presents an OCT scanning system 102 according to some embodiments of the present invention. System 102 is similar to system 101 and differs therefrom in that ultrasound transducer 134 is a rectal ultrasound probe in this case, and comprises a needle guide 77 which passes through the body of transducer 134. (Compare to an ultrasound system shown in FIG. 3, which utilized a needle guide 76 external to the transducer.)

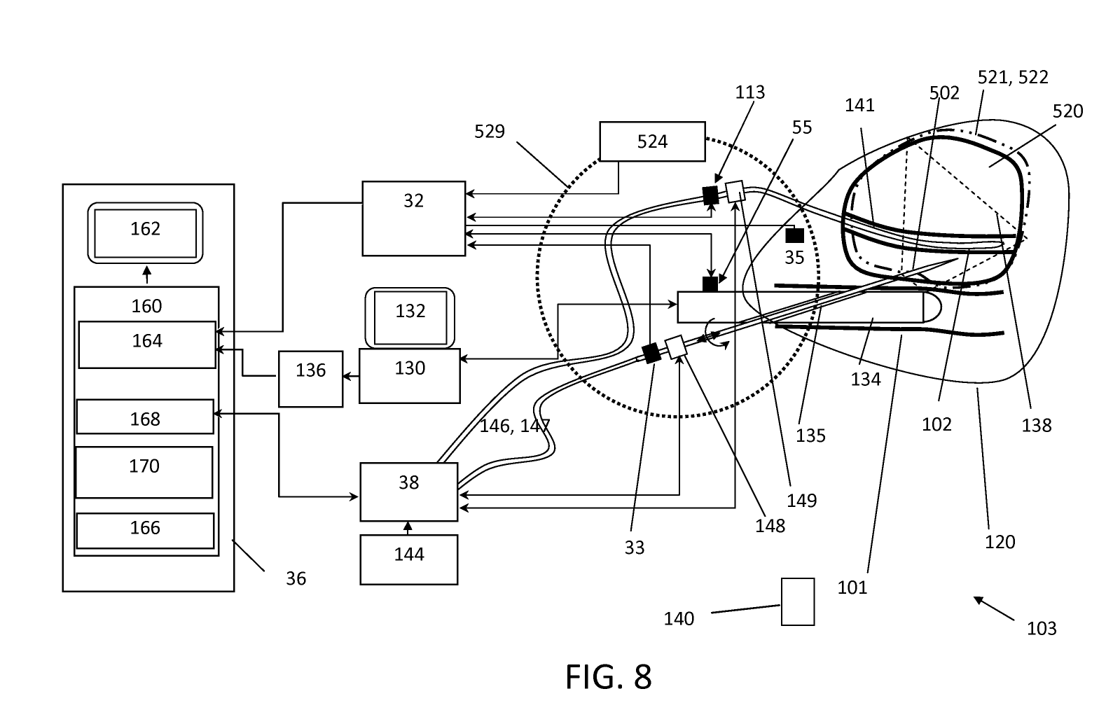

Attention is now drawn to FIG. 8, which presents an OCT scanning system 103 according to some embodiments of the present invention. System 103 is similar to system 102 and differs therefrom in that system 103 comprises a catheter-based OCT probe introducible into a urethra of a patient by means of catheter 141. Information from an OCT probe of catheter 141 may also be integrated into mapping 522 and model 521 by processor 160, along with that of a probe 502 introduced into the prostate through transducer 134 inserted in the anus.

FIG. 8 also shows additional sensors reporting to probe location system 300, sensor 55 reporting on position of catheter-based OCT probe 141 and sensor 113 reporting on location of ultrasound transducer 134, helping thereby to integrate ultrasound images with OCT-scanned information, as discussed above.

FIG. 8 also shows a probe actuator 149, which is similar to probe actuator 148 but is designed to work with catheterized probe 141, to which it imparts longitudinal (514) and rotational (515) movements required for scanning.

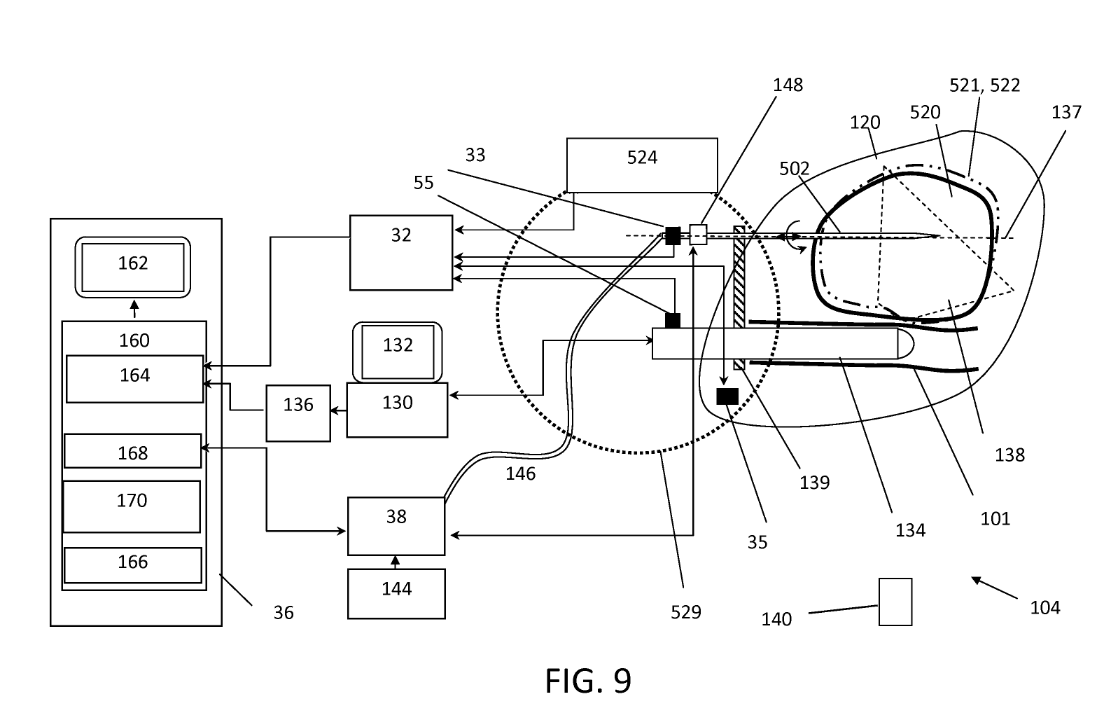

Attention is now drawn to FIG. 9, which presents an OCT scanning system 104 according to some embodiments of the present invention. System 104 is similar to system 103 and differs therefrom in that system 104 comprises a template 139 which comprises a plurality of guiding slots for guiding a plurality of OCT probe insertions into a plurality of positions within an organ 520. Template 139 could be used, for example, to guide a series of insertions of OCT probes into a prostate through the perineum.

Attention is now drawn to FIG. 10, which is simplified schematic of a rotating OCT probe, according to some embodiments of the present invention. FIG. 10 presents a probe 502. The embodiment shown in the figure is also labeled 802, and comprises two concentric tubular devices, outer tube 210 being able to remain stationary during scanning, while inner tube 212 rotates. Probe 802 optionally comprises cylindrical window 214 attached to outer tube 212. Window 214 enables 360° radial scanning, because light beams may be sent from probe 802, and that light, reflected and scattered light from tissues, may return to probe 802 through window 214 and then be used for optical coherence tomography analysis and image detection. Probe 802 also optionally comprises, at its distal end, a sharp end shape 211 (e.g. a conical shape as shown in the figure) which facilitates penetration of probe 802 into tissue. Optionally, sharp end shape 211 may be formed as a transparent window to allow scanning therethrough, and may optionally be continuous with or optionally be provided instead of, window 214.

Outer tube 210, optionally constructed of metal or a similarly hard material, transports and protects inner rotating tube 212.

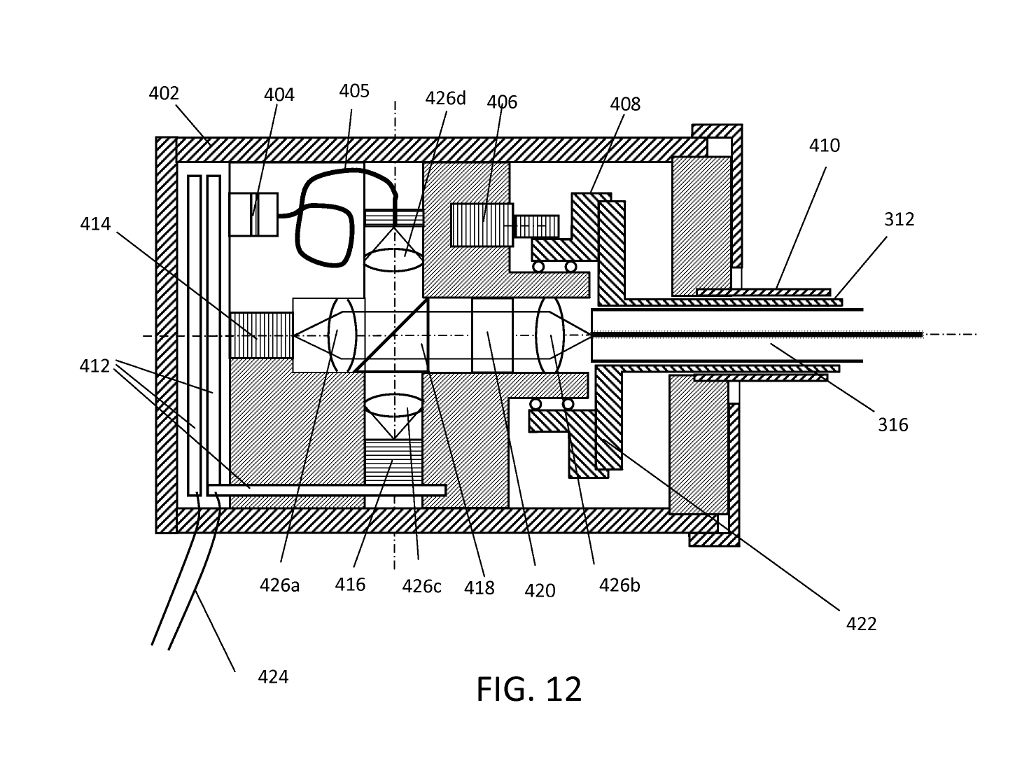

In use, probe 802, optionally with sharp distal end 211 forward, is optionally inserted into a tissue to a desired depth. Insertion may optionally be guided by ultrasound or by another imaging modality, such as fluoroscopy, CT or MRI. In an optional mode of operation an operator or a probe-positioning servomechanism slowly withdraws probe 802 while rotating tube 212 using a circular scan motor 406 (shown in FIG. 12). Each complete rotation of tube 212 produces a ‘slice’ image optionally reported to processor 160 as part of imaging data stream 168.

Probe 802 differs from, for example, probes disclosed by Pitris op. cit., inter alia in that probe 802 comprises a position tracking sensor 33. Sensor 33 may optionally be mounted on probe 802 or may optionally be embedded within the structure of probe 802. As explained above, sensor 33, part of location tracking module 300 enables calculating the spatial locations of objects imaged by the probe.

Outer diameter of probe 802 in some embodiments is between 0.5 mm and 3 mm. Length of probe 802 in some embodiments is between 20 mm and 150 mm. Rotating portion 212 comprises optical fiber bundle 200, optionally contained in a tube as shown in the figure, a lens 217, and a beam director 218 optionally attached to lens 217.

Rotating portion 212 is optionally able to move distally and proximally (i.e. advancing and retracting within probe 802) and can rotate inside external stationary portion 210. These movements and their role in scanning tissue were explained above, inter alia with respect to FIG. 1B.

In a proximal portion of probe 802 a mechanical translation & rotation element 148 is optionally controlled by OCT console 38 (shown in FIG. 4). An optical fiber cable 146 is provided to communicate light signals to and from body tissue via beam director 218. Scanning of a tissue adjacent to probe 802 is accomplished by acquiring depth information along the beam direction 216, and by rotating and advancing/retracting internal assembly 212 using the translation & rotation element 148. Additionally, probe 802 may be advanced and/or retracted as a whole, stepwise and/or continuously, to bring probe 802 to bear on additional portions of tissue along probe 802's insertion path.

Optionally, a plurality of windows 214, optionally of different shapes, may be mounted at different positions along or around probe 802, enabling moveable portion 212 to interact with tissue from a plurality of different positions, without necessarily advancing or retracting probe 802 as a whole. Optionally, sharp head 211 may be formed of transparent material and may function as head 211 and as window 214 also.

Attention is now drawn to FIGS. 11A-11C, which are views of an OCT probe 803 which comprises a sharp tip 311 attached directly to a rotating assembly 312, according to some embodiments of the present invention.

As shown, rotatable inner tube 312 holds optical fiber cables 316 (core) and 302 (clad). Near a distal end of probe 803 fiber optic cable 316 ends at a focusing lens 317 (such as a GRIN) and a reflector (beam director) 318. Beam director 318 serves to direct a light beam from fiber optic 316 laterally, sending the beam in a radial direction.

The configuration of probe 803 may help to protect optical windows 306 of probe 803 during insertion. Base 422 of rotatable assembly 312 connects to motors which induce rotational and/or longitudinal motions of assembly 312 within outer tube 300. (The motors are not shown in the figure.) Assembly 312 can be advanced and retracted within metallic outer sheath 300, as may be seen schematically in FIGS. 11B and 11C. In an optional method of use, during insertion probe 803 is positioned in a configuration shown in FIG. 11B, where transparent window 306 is protected from abrasion and from contact with obscuring material. For scanning, assembly 312 may be advanced to a position shown in FIG. 11C, exposing transparent window 306 to the surrounding tissue.

FIG. 11A further discloses two optional subassemblies which may help keep window 306 transparent. They are an injection channel 318 and a wiper 312.

Injection channel 318 may be used to inject fluids 319 into probe 803, optionally for cleaning window 306 or for other purposes. Transparent fluorocarbon blood substitutes may be used in this context as fluid 319, and can literally wash window 306 of blood or other obscuring material. Sealing elements (e.g. O rings) 307 cause injected fluid 319 to flow forward between outer sheath 300 and inner assembly 312, forcing fluid 319 to emerge next to window 306, cleaning it.

Edge wiper 312, shown in the inset in the upper right corner of FIG. 11A, contacts window 306 when assembly 312 is moved proximally or distally (in and out of the protecting shell 300) and functions rather like a windshield wiper, cleaning window 306.

Probe tip 311 optionally provides a sharp distal end, enabling probe 803 to move distally and penetrate tissue. Tip 311 is optionally made of metal or ceramic or other suitably hard material. Optional alternative tip 320 is transparent, and fulfills the functions of both window 306 and sharp tip 311.

Attention is now drawn to FIG. 11D, which is a simplified schematic showing an addition use for probe 803, according to an embodiment of the present invention. In FIG. 303, internal assembly 312 has been entirely retracted from outer body 300 of probe 803. Once this has been done, external portion 300 may serve as a cannula for guiding an additional operative needle into tissues. In an optional method of use, probe 803 may first be used to identify and diagnose abnormalities or illness of the imaged tissue surrounding the probe. Once a suspected tissue is identified, assembly 312 may be removed and an alternative operative device 350 may be inserted in its place, arriving among tissues which optionally have been scanned by probe 803 and whose structure, including positions of lesions, is well known. Any appropriately shaped operative device 350 can then be inserted through body 300, for additional diagnostic operations or for a therapeutic procedure. For example, device 350 might extract blood, deliver a fluid, plant radioactive seeds, coagulate tissue, or cool tissues to cryoablation temperatures.

Attention is now drawn to FIGS. 11E and 11F, which are views from above and from the side respectively of an additional embodiment of probe 803. An optional probe head similar to head 320 of FIG. 11A and here labeled 720, comprises a slot 701 for taking a biopsy sample. When inner portion (e.g. assembly 312) is advanced, advancing head 720 into tissue, some tissue may enter slot 701. When head 720 is then retracted into body 300 of the probe (with body 300 optionally advancing simultaneously, so that slot 701 may remain with its inserted tissue 702 still in slot 701. An advancing portion of body 300 may then cut off a portion of tissue 702, trapping it in slot 701 and protecting it from change while probe 803 is optionally retracted from the body, bringing with it biopsy sample 702. The embodiment shown in FIGS. 11E and 11F is consequently an OCT probe which is also a biopsy needle. It is noted that an embodiment according to FIGS. 11E and 11F may be used with a system embodiment operable to detect a lesion in tissue, as discussed above. Some embodiments of a method of using an OCT probe to detect a lesion, and subsequently using that OCT probe to take a biopsy sample of tissue of said lesion.

Attention is now drawn to FIG. 12, which discloses a miniature interferometer incorporated directly on an OCT probe (for example, optionally, incorporated in probe 803 discussed above) and encapsulated into a handset housing 402 of an OCT probe, according to an embodiment of the present invention. Either a Michelson interferometer or a fiber interferometer, both known in the art, can be incorporated in housing 402.

A rotation motor 406 is provided for rotating inner probe tube 312 via a rotating cap 408 and rotating assembly base 422, rotating fiber core 316 inside protecting sheath 312, and rotating other distal parts as described above. The assembly is then able to perform a 360° scan of tissues as described above. Illumination is provided by source 414, optionally a partly coherent super luminous diode (used for operating in time domain configuration) or optionally a monochromatic scanned source (used for operating in the Fourier domain configuration). Optionally, miniature PCBs 412 control scanning motor 406, power to light source 414 and timing of light pulses, movement of scanning minor 404, and signals and data from detector 416. An interferometer adopted for installation within handset housing 402 (internal to and OCT probe) includes moving mirror 404, internal optical fiber 405 (with optical path similar to the optical path of the OCT probe), detector 416, lenses 426a, 426b, 426c, 426d, TC lens 420, and beam splitter 418.

In some embodiments, to reduce friction of rotating parts, some surfaces may be coated with a friction reducing layer such as hydrophilic coating. Optionally, the gap between rotating tube 312 and stationary tube 300 may incorporate a friction-reducing spacer made of Teflon or an equivalent material.

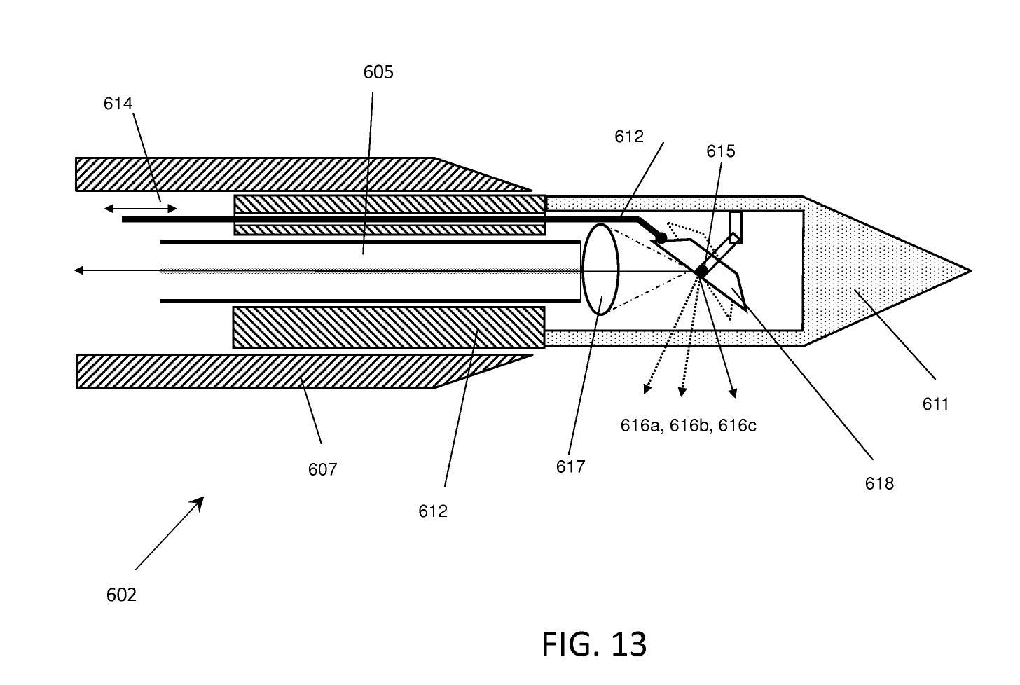

Attention is now drawn to FIG. 13, which is a simplified schematic of an OCT probe 602 which comprises a tiltable beam director 618, according to some embodiments of the present invention. Beam director 618 provides a scanning option not available from probes known in prior art: scan light may be directed in directions and in patterns which are impossible to achieve with previously known OCT probe designs.

Like OCT probes 502 and 802, OCT probe 602 has internal moving/rotating parts, including internal optical fiber bundles 605, lens 617, and beam director 618. Probe 602 also comprises an outer tube 607. Probe 602 optionally comprises a tip 611, which is optionally optically transparent.

Probe 602 comprises a tiltable beam director 618 which enables to direct a laterally directed OCT beam to a plurality of different directions, shown in the figure as directions 616a, 616b, and 616c. A lever 612 may pulled or pushed as shown by arrows 614, and used to steer beam director 618 on pivot 615, thereby steering beam director 618 inward and outward. Steering lever 612 may optionally be manually operated and may optionally be operated by a motion controller (e.g. a probe positioning module 140) and may be connected to OCT console 38. Because of the additional degree of freedom available in operating probe 602, OCT scan data may be generated using probe 602 in patterns not available using OCT probes known to prior art.

It is expected that during the life of a patent maturing from this application many relevant OCT technologies will be developed and the scope of the terms “Optical Scanning Tomography” and “OCT” are is intended to include all such new technologies a priori.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1-50. (canceled)

51. A system for creating a three dimensional map of at least a portion of an organ, the system comprising:
at least one Optical Coherence Tomography (OCT) probe configured to report imaging data while inserted in said organ; and
a processor programmed to receive said imaging data during a plurality of tissue insertions of said at least one probe and to record said data with reference to a three-dimensional coordinate system.

52. The system of claim 51, wherein said data extends over a three-dimensional volume greater than a volume imageable by said OCT probe during a single tissue insertion of said plurality of tissue insertions.

53. The system of claim 51, further comprising a probe location module configured to report a location of said at least one OCT probe while said probe is reporting imaging data, wherein said probe location module utilizes a sensor reporting the location of said OCT probe.

54. The system of claim 51, further comprising a probe positioning module configured to position said OCT probe at a selected position according to a received command specifying said selected position.

55. The system of claim 51, further comprising a template which comprises a plurality of guiding channels for guiding said OCT probe during said plurality of tissue insertions.

56. The system of claim 51, further comprising a second imaging modality in addition to said OCT probe, wherein said second imaging modality is configured to report a location of said at least a portion of said organ to at least one of:
a processor; and
a display visible by a user.

57. The system of claim 56, further comprising a position reporting module configured to report a position of said second imaging modality during imaging operation of said second imaging modality, wherein position reporting module comprises a position sensor reporting the location of said second imaging modality.

58. The system of claim 56, wherein said second imaging modality is an ultrasound probe which comprises a guide configured to guide said plurality of tissue insertions.

59. The system of claim 51, wherein said processor is further programmed to analyze image data reported by said OCT probe and to detect; based on said data, an imaged border of said organ.

60. The system of claim 51, further comprising a display for displaying an image based on at least a part of a three dimensional mapping created by said system.

61. The system of claim 60, further comprising a display calculation module configured to calculate a view based on information from said three dimensional mapping, which information was at least partially calculated based on some of said imaging data.

62. The system of claim 60, further comprising a display calculation module configured to calculate a view based on information from said three dimensional model, based on information from OCT scanning and information from at least one of:
a historical data source; and
an additional imaging modality, other than OCT scanning.

63. The system of claim 62, wherein said calculated view is based on information received by said processor during said plurality of tissue insertions.

64. The system of claim 62, wherein said calculated view is a slice image of a portion of said organ.

65. The system of claim 62, wherein said display calculation module is further configured to calculate a view based OCT scan data and on at least one of a group consisting of:
information from a historical source; and
information from an imaging modality other than said OCT probe.

66. A method for creating a three dimensional map of at least a portion of an organ, the method comprising:
performing a plurality of insertions of at least one Optical Coherence Tomography (OCT) probe into tissue at a plurality of sites, each site differently positioned with respect to said organ; and
using a processor to create a three-dimensional mapping of said at least a portion of said organ based on image data reported by said at least one OCT probe during said plurality of insertions.

67. The method of claim 66, further comprising using a probe location module to report to said processor locations of said at least one OCT probe during said imaging during said plurality of insertions.

68. The method of claim 66, further comprising using said processor to calculate, as a function of said imaging data and of information relation to position of said at least one probe during said imaging, a position of an imaged feature in three-dimensional space.

69. The method of claim 66, further comprising using said processor to analyze image data from said probe to detect at least one of
imaging of a border of said organ; and
imaging of a lesion in said organ.

70. A method for three-dimensional (3D) mapping of a region of interest in a body, the method comprising
using a probe positioning module to insert an Optical Coherence Tomography (OCT) probe into a plurality of probe insertion sites within a region of interest in a body; and
using a 3D mapping module to calculate a 3D model of said region of interest, said calculation being based at least in part on:
a first data stream reporting positions of said OCT probe during said insertions at said plurality of probe insertion sites, and
a second data stream comprising imaging data generated by said OCT probe during said insertions at said plurality of probe insertion sites.
